(19) United States
(12) Patent Application Publication
(10) Pub. No.: US 2013/0108667 A1
(43) Pub. Date: May 2, 2013

(54) Method, apparatus and system for electroporation

(71) Applicants: Soiwisa Soikum, Sri Ayutthaya (TH); Lars Thomsen, Ipoh (MY); John Robert Dodgson, London (GB)

(72) Inventors: Soiwisa Soikum, Sri Ayutthaya (TH); Lars Thomsen, Ipoh (MY); John Robert Dodgson, London (GB)

(21) Appl. No.: 13/317,739

(22) Filed: Oct. 27, 2011

(51) Int. Cl. A61B 18/00; C12M 1/42; G01N 27/00; A61K 39/395; A61K 38/02; A61K 31/7088; A61K 48/00; C12N 13/00; A61K 9/00; B82Y 5/00

(52) U.S. Cl. 424/400; 606/41; 435/173.6; 435/283.1; 435/7.2; 435/29; 424/130.1; 514/1.1; 514/44 R; 977/773; 977/902; 977/915

(57) ABSTRACT

A method, apparatus and system that employs particles, e.g., nanoparticles, and an electric or electro-magnetic field, to cause electroporation in target cells at reduced fields. Electroporation may be irreversible, leading to targeted cell death, or reversible, allowing species to be introduced into the target cell. The method introduces a particle to a position adjacent to the cell membrane of a target cell and exposes the target cell to a transient electromagnetic field for a time interval to cause targeted electroporation. A smaller electric field is applied, thereby surmounting similar methods. The particle enhances the effect of the electric field in its immediate vicinity, so reducing the field strength needed to achieve electroporation and thereby reducing the risk of damage to cells through high field exposure. Electroporation can be targeted to a subset of target cells by targeting the particles to surface markers on the target cell membrane.

### FIELD OF THE INVENTION

The present invention relates to a method, apparatus and system for electroporation of cells. More particularly the invention relates to a method, apparatus and system that employs particles, or nanoparticles, together with an electric or electro-magnetic field to enhance electroporation in target cells. Electroporation may be reversible, or may be irreversible, so as to cause death of the target cells.

1. Background of the Invention

The use of electro-magnetic fields for treatment of cancer by causing lysis to cells that harbor, or are in close proximity to particles responsive to such fields, as a result of local heat deposition within or adjacent to the cells, is known. The techniques involve using a micro sized particle, typically below 0.1 μm, which for example comprises an iron oxide core and is coated with a polymer. Typically a secondary coating is applied where the particle is activated by chemicals that show specificity for biological targets of interest. The aim is to concentrate particles at the site of a biological target, where a specific chosen target can be an exclusive marker, expressed for example by cancer cells, and presented on the extracellular side of the cell. Target cells are then destroyed thermally by means of absorption of energy from the electromagnetic field.

Efforts are being made to improve techniques by focusing on factors, such as clearance of particles from blood before reaching the target cells; binding unselectively to other than target cells; toxicity of the particles in use and insufficient thermal effect from induced electric fields to cause killing of the target cells. In particular, particle-mediated thermal killing of target cells has the disadvantage that the locally generated heat is readily conducted away from the site of generation, leading to ineffective killing of target cells and damage to neighbouring healthy cells, and the need for high RF power densities in treatment.

Irreversible electroporation (IRE) is known as a means for non-thermal destruction of cells by exposing them to a field above the threshold field at which pores formed in the membrane in the applied field do not re-close, leading to cell death. Irreversible electroporation has been used in treatment of diseases, such as cancer.

Reversible or non-invasive electroporation of cells, in which an electric field applied to a cell causes the formation of pore's in the cell membrane through which an entity or substance to be introduced into the cell may pass, the pores then resealing with retained viability of the cell, is a known technique. However, the field needed to achieve pore formation is high and needs high potentials to be applied to electrodes placed close to the cells in order to succeed. While routinely used for in-vitro processes such as transfection of cells, electroporation treatment of cells in-vivo for treatment of disease is still accompanied by problems, partly owing to the high fields that are needed and difficulty in ensuring the threshold field for electroporation is reached in all desired cells, in a region of tissue that will of necessity experience a non-uniform field intensity owing to variable degrees of screening of cells by other cells. The field needed to achieve high efficiency of electroporation frequently exposes a proportion of the cells to a field above the threshold for irreversible electroporation, leading to loss of viability of the cells or tissue.

In particular, electroporation has been proposed to introduce diagnostic or therapeutic entities into cells, such as for example drugs to kill cancer cells or particles small enough to enter the cells through the pores, which may be themselves carriers of drugs or have therapeutic action, for example in phototherapy or magneto-thermal therapy for cancer.

However, the problem of the high field required for both reversible and irreversible electroporation, and hence the need in many applications for invasive electrodes planted close to the cells of interest, and the potential problem of skin burns owing to a high voltage drop if external electrodes are used, has limited progress in these applications. A further problem has been the lack of ability to target electroporation to a specific target cell type within a mixed population—for example, a cancer cell within healthy tissue.

It is known in the prior art that the effect of the field may be enhanced by Carbon Nanotubes (CNT) associated with the cell membrane, and that electroporation may therefore occur at lower field than in the absence of CNT. It has also been proposed in the prior art that particles may be targeted to specific cells for the purpose of treatment of cancer by means of irreversible electroporation, and that such particles may be CNT or microspheres.

The present invention provides improvements to methods, apparatus and systems for use of particles to enhance electroporation, including irreversible electroporation for targeted killing of cells.

2. Prior Art

The use of CNT as mediators to reduce the threshold for reversible electroporation is disclosed by Rojas-Chapana et al (Lab Chip 2005, vol 5, 536-9), in which plasmid DNA was introduced into E coli cells by 2.45 GHz microwave irradiation in the presence of CNT, with no effect in their absence.

International Patent Application WO-A-2008/062378 (Raffa et al) discloses the use of carbon nanotubes to reduce the field at which reversible electroporation occurs. In Raffa et al the electroporation field is applied by a pair of parallel plate electrodes and it is claimed that the effect depends on the orientation of the CNT: orientation with the axis of the CNT parallel to the field and perpendicular to the membrane is stated to be disadvantageous for eukaryotic cells, and Raffa et al disclose a second pair of electrodes applying a field in a direction orthogonal to the electroporation field to align the CNTs with their axis parallel to the cell membrane. Raffa et al show an in-vitro example of the effect of CNTs used in this way but this is clearly cumbersome to arrange for in-vivo treatment of disease. Additionally, CNTs have been shown to be cytotoxic and are therefore potentially unsuited for applications in-vivo.

Applicant's co-pending International Patent Application PCT/GB2011/000645 discloses a method and apparatus for killing cells, and treatment of disease such as cancer or pathogenic infections, involving targeted destruction of cells by irreversible electroporation at a reduced applied field through enhancement of the electric field experienced by the cell membrane, by means of dielectric or conducting particles, in particular gold nanospheres or Fe3O4-cored nanoparticles, located on or adjacent to the cell membrane. Field enhancement resulting from the interaction of a pair of particles was disclosed, and the application discloses the use of pairs of particles to achieve irreversible electroporation, with a preferred arrangement of a particle associated with the exterior of the cell membrane and a further particle inside the cell. It was disclosed that one or both particles may comprise a coating selective for a target molecule present on the surface of a target cell, so allowing irreversible electroporation to occur selectively for the target cell in a mixed population, for example for cancer cells while healthy cells are left undamaged.

International Patent Application WO-A-2010/151277 (Davalos et al) discloses a method of treating neoplasia in a subject using irreversible electroporation in which nanoparticles are administered to the subject in an amount sufficient to permit at least some of the nanoparticles to come into close proximity to the neoplastic cells. At least two electrodes are implanted into or adjacent the neoplastic cells within the body of the subject. WO-A-2010/151277 discloses that the use of nanoparticles can reduce the threshold field for the destruction of cells by means of irreversible electroporation, and so allow an electric field to be chosen such that cells having nanoparticles in proximity are destroyed while those without particles remain are not destroyed. The use of fields in the range between 500 and 1500 V/cm is specified. If the nanoparticles have a coating that allows them preferentially to associate with a neoplastic cell, rather than healthy cells, this allows the neoplastic cell to be destroyed while the healthy cells are undamaged.

WO-A-2010/151277 further discloses that when the field is applied a volume of cells outside the region in which irreversible electroporation occurs may experience a field lower than the threshold for irreversible electroporation, and may undergo reversible electroporation, so that use of nanoparticles comprising a drug, such as an anti-cancer drug, may increase the volume that is treated for any given electrode placement. As reversible electroporation is mentioned briefly and only as a side effect of the irreversible electroporation procedure, no disclosure is made of the method or apparatus for achieving reversible electroporation of cells, or reversible electroporation of a region of cells in the body without concurrent irreversible electroporation in a neighbouring region.

The inventors have now improved methods, systems and apparatus for both irreversible and reversible electroporation using particles as mediators.

Soikum and Thomsen disclosed an improved method for destruction of cells by irreversible electroporation in a poster, published at the Nordic Naiad Symposium, Copenhagen, from 1-3 Nov. 2010. The publication discloses a field enhancement effect that arises as a result of use of a nanoparticle of high electrical permittivity, or high conductivity, for example that obtained when using a metal nanoparticle, adjacent to the exterior of a cell membrane. The publication illustrated field enhancement by a nanoparticle adjacent a cell membrane, and the publication disclosed the application of particle mediated cell destruction in killing bacteria in-vivo, in treatment of human diseases and in agricultural applications. The use of nanoparticles to enhance field flux and achieve cell destruction in-vivo using an external field generator was disclosed. Particles having a high electrical permittivity, in particular particles having a high permittivity relative to that of blood (with relative permittivity 88), and conductive (for example, metal) particles, were disclosed as being suitable for application of the invention in-vivo.

Additionally, a field enhancement effect was shown theoretically for a pair of gold nanospheres in proximity to each other in a medium with the same salt concentration as human blood, with the field applied by a pair of electrodes external to the solution, representing the situation of a pair of electrodes external to the cellular environment in-vivo. The field enhancement was greatest in the region between the particles. The diagram shown in this disclosure is reproduced herein as FIG. 5a.

The inventors now describe further the method above and describe apparatus and systems adapted for carrying out the method.

### DETAILED DESCRIPTION OF THE INVENTION

According to a first aspect of the invention there is provided a method of causing electroporation of a target cell comprising the steps of: providing one or more particles associated with the exterior of the cell membrane of a target cell and exposing the target cell to a transient electric field for a sufficient time interval in order to cause electroporation of the cell.

As a result of introducing the particle to the exterior of the cell, it has been found that when an electric field is applied the particle enhances the effect of the electric field in the vicinity of the particle, so reducing the field strength needed to achieve electroporation of the cell.

In a preferred embodiment in which irreversible electroporation is desired, the applied field is selected such that the enhanced field across the cell membrane is greater than the threshold for irreversible electroporation, so causing cell death.

In a further preferred embodiment in which reversible electroporation is desired, the field applied is selected such that the local field across the membrane in the vicinity of the particle is greater than the threshold field for reversible electroporation and less than the threshold for irreversible electroporation.

Preferably the particle is a dielectric particle. Preferably the particle has a high permittivity or conductivity with respect to the cell and a surrounding environment. In some embodiments the particle is conductive or comprises a conductive core, such as a metal, for example gold, or a conductive or semiconductive metal oxide.

In preferred embodiments the particle has a low aspect ratio, as compared with for example a carbon or boron nanotube as used in the prior art. By aspect ratio is meant that the ratio of the maximal to the minimal dimension of the particle, for example the ratio of length to diameter, which for a CNT may typically be 50:1 or more. CNT are known in the art to be cytotoxic, and that this is related to their morphology—they are taken up by cells by endocytosis, but are not completely engulfed, which leads to destruction of the cell. This is potentially a source of toxicity to a subject undergoing treatment for disease.

Raffa et al disclose the further disadvantage of an axial orientation of CNT in that the field enhancement at the end of the tube may lead to destruction of the cell in the case that the cell is a eukaryote cell. Therefore in preferred embodiments of the present invention the particles have low aspect ratio, preferably an aspect ratio of less than approximately 50, more preferably less than approximately 10. In preferred embodiments the particles may comprise substantially spheroidal particles, for example gold nanospheres, approximately spherical iron oxide nanoparticles or polygonal particles with varying geometry, for example particles comprising semiconductor materials such as quantum dots. Elongated particles, such as gold or iron oxide nanorods with aspect ratio approximately 20 or less, do not have apparent toxicity problems and such particles are employed in a preferred embodiment, and are referred to herein as low aspect ratio particles.

In preferred embodiments the particle is ideally associated with the cell membrane, or in the case of a bacterium, the cell wall. The particle may be adapted to bind or adhere to the cell wall.

In preferred embodiments the electric or electromagnetic field is applied to the cell by means of potentials applied to a first and a second electrode remote from the cell. The field between the electrodes is enhanced in the vicinity of the particle, so providing an enhanced field across the cell membrane, such that in some embodiments while the externally applied field may not reach the threshold for electroporation, the enhanced field is above the threshold.

The local field strength at the membrane surface needed for irreversible pore formation in a cell membrane depends on the cell type and its surrounding environment, but it is known in the art, and stated by Davalos et al., to lie in the range approximately 500V/cm and above, typically in the range 500 to 1500V/cm.

The local field strength at the membrane surface needed for reversible pore formation in a cell membrane also depends on the cell type and surrounding environment, and is known in the art typically to lie in the range 50 to 500V/cm.

Therefore in a preferred embodiment directed to irreversible electroporation the method of the invention comprises applying time varying potentials to at least a first and a second external electrodes sufficient to achieve a local field at the cell membrane, in the presence of a particle associated with the membrane acting to enhance the applied field, in the range 500V/cm and above.

In a further preferred embodiment, directed to reversible electroporation, the method of the invention comprises applying time varying potentials to at least a first and a second external electrodes sufficient to achieve a local field at the cell membrane, in the presence of a particle associated with the membrane acting to enhance the applied field, in the range 50 to 500V/cm.

The externally applied field needed to achieve this local field strength depends on the cellular environment, for example whether in suspension or in-vivo and, in-vivo, the location of the cell, the distribution of body fluids and tissue in the surrounding environment, and the dimensions of the tissue or body region between the electrodes and the target cell(s). Therefore the externally applied field and the potentials at the electrodes needed to apply it are dependent in part on the application and, in-vivo, the indication, and the location of the target cells to be electroporated within the individual subject. The applied field and electrode potentials are selected in order to achieve an optimum degree of effect, either for irreversible or for reversible electroporation. It will be understood from the above that a certain applied field in a given application may cause reversible electroporation and in another application may cause irreversible electroporation, and in preferred embodiments the method comprises selecting the field accordingly for the desired effect.

The field enhancement effect of the particles allows the applied field, as measured between the first and second electrodes, to be lower than the desired local field adjacent to the particle and membrane. The field enhancement effect may depend on the number and location of particles within and adjacent to the target cells.

In a typical embodiment in which particles adjacent to the cell membrane enhance the applied field, applied fields above approximately 500V/cm are likely to cause irreversible electroporation, applied fields between approximately 100V/cm and 500V/cm may cause irreversible electroporation in certain cell types and configurations of particles and cellular environments, and reversible electroporation in others, while applied fields below 100V/cm are more likely to cause reversible electroporation for a range of cell types and configurations. Therefore the method includes that the applied field parameters may be selected in order to optimize the electroporation effect, either irreversible or reversible, in dependence on factors including the cell type, the cellular environment, the type, quantity and intended location, within or bound to the target cell, of particles used in the method.

Therefore in embodiments directed to irreversible electroporation the maximum applied field lies preferably in the range below 1500V/cm, more preferably between 1000V/cm and 500V/cm, more preferably still between 500V/cm and 100V/cm, and in some embodiments below 100V/cm.

In embodiments directed to reversible electroporation the maximum applied field preferably lies in the range below 500V/cm, more preferably between 500V/cm and 100V/cm, more preferably still between 100V/cm and 20V/cm, and in some embodiments below 20V/cm.

It is a feature of the invention that in a given application the applied field needed for successful electroporation depends on the cell type, cell density and the configuration of the cells and surrounding fluids, the number and type of the particles present, and the dimensions and configuration of the electrodes. It is envisaged that in some embodiments the method of the invention comprises a procedure in which a set of field parameters (field strength, pulse shape and repetition etc.) is selected, electroporation according to the method is carried out, the degree of electroporation, either irreversible or reversible, is assessed, the field parameters are changed, and the process is repeated in a feedback process, which may be manual but in some embodiments may be automatic, for example under the control of a control means, until successful parameters are achieved. An advantageous feature of the invention is that the applied field that is needed is reduced, allowing lower potentials to be used, or in some embodiments electrodes to be used external to the body of a subject undergoing in-vivo electroporation treatment rather than implanted as in the prior art.

The terms electric and electromagnetic fields are used interchangeably herein except where stated. The terms electric field, electric field strength and electric field flux are also used interchangeably to refer to the magnitude of an electric field. An applied field may be defined in terms of the magnitude, polarity and time course of the field. By selection of the applied field or applied electromagnetic field as used herein is meant the selection of the maximum or peak field strength, the polarity and the time course of the field, by means of parameters such as the magnitude of a pulse, pulse shape, rise and fall times, polarity, repetition rate and total number of pulses of a pulsed field, and the peak value, waveform, frequency, swept change in frequency and duration of an AC field.

The electric field is ideally a time varying field but it will be understood that alternatively, or in addition, the electric field may vary in space, so that a varying field gradient is applied to the targeted cell.

According to a further aspect of the invention particles may have formed thereon a coating that makes them selective for the target cell type in such a way that at least one particle binds selectively to one or more target molecules in the target cell membrane, expressed preferentially or exclusively by the target cells.

By this means target cells, as they have at least one particle associated with them, undergo electroporation while non-target cells, without associated particles, do not. Such coatings allow the method of the invention to be applied selectively to target cells in a mixed population of target and non-target cells, allowing the field to be chosen so as to cause electroporation of the target cells while leaving the non-target cells unchanged.

Above a higher field threshold cells undergo irreversible electroporation and die. In conventional electroporation the aim is to provide a local field at the cell membrane above the electroporation threshold but below the irreversible electroporation threshold. In practice the window between the two thresholds can be narrow, resulting in irreversible electroporation of a proportion of the cells.

The method of the invention allows reversible electroporation of target cells at low applied field strength, while non-target cells without particles experience a field well below the threshold for irreversible electroporation, and so the chance of damage to non-target cells is reduced.

The term target molecule means any molecule, or region or fragment of a molecule, for example a protein, present on the exterior or interior of a target cell to which a coating, component or region of a particle may associate or bind. The target molecule may be a biomarker and the terms marker and target molecule are used interchangeably.

Target molecules may be the following or regions or subunits of them: lipid, carbohydrate, a nucleic acid (such as chromosomal DNA and/or plasmid DNA and/or any type of RNA), a protein (for example, from the group comprising: enzymes, structural proteins, transport proteins, ion channels, toxins, hormones, and receptors) or small molecules that can be bound to the cellular membrane either in form of an agonist and/or antagonist compared to its affinity for non-target cells.

In a preferred embodiment, such target cell molecules are located on the exterior of the cell membrane so providing one or more particles bound preferentially to the exterior of the target cell, but not to the exterior of non-target cells. Such targeting may be achieved by means of antibodies, aptamers or other ligands provided as part of the coating on the surface of the particles. Ligands for known target molecules as described in the prior art may be used in the coating. More than one ligand species may be provided in order to increase the capture affinity.

Such a coating comprising ligands may be uniform over the surface of the particle or it may be located in a specific region of the particle, for example in the case of an elongated structure, preferentially at one end of the particle or preferentially remote from one end, so allowing the particle to bind preferentially in a preferred orientation with respect to the target cell.

In embodiments directed to reversible electroporation, the method may be applied to enable an entity or substance to be introduced into the cells to diffuse through the temporarily open pore(s), or to move electrophoretically under the influence of the field, or both. The term species will be used herein to describe entities such as particles as well as chemical substances, and may comprise for example a drug, a protein, a nucleic acid such as DNA or RNA, for example a genetic construct as used in transfection of cells or RNA construct used in for example gene silencing. The substance may be in ‘naked’ form, encapsulated in a shell or coating, or bound to a further entity such as a particle. The species may be for therapeutic or diagnostic effect, for example markers or sensor particles, for example fluorescent beads or quantum dots, coated, functionalized or targeted by means of a ligand coating on the particle.

In a preferred embodiment the invention comprises particles adapted to associate with the cell membrane in order to enhance the local field, further adapted to enter the target cell as part of the reversible electroporation process. In such embodiments the particles comprise a coating adapted to facilitate this, comprising a ligand species having a binding affinity selected to allow the particle to dissociate from the target molecule in order to enter the cell. The particle is adapted to have a desired functionality within the target cell, such as to form a marker particle or to deliver a desired payload to the cell. The particle preferably further has a coating comprising a payload to be delivered to the target cell, for example of types described above and as known in the art. In some embodiments the coating may further comprise ligand species adapted to associate with target molecules within the target cell.

According to a further aspect of the invention the method further comprises a step of introducing one or more particles to the inside of the target cell prior to application of the field. The particle within the cell acts to enhance the effective electric field across the membrane.

It is known in the prior art that small particles are taken into cells by means of endocytosis and the invention includes the use of particles adapted to promote endocytosis. Such adaptation may also include providing a surface coating or surface species that promote uptake by cells. Such a coating may include surface molecules such as protein or peptide species known in the art to promote endocytosis. Such a coating may comprise species such as proteins, peptides or nucleic acid species that bind to target molecules on the exterior of the cell membrane, so acting to increase the concentration of particles associated with the membrane, and hence the rate of uptake of the particles by means of endocytosis, even when not specifically caused or enhanced by the coating of the particle.

The particles may be adapted to bind to target molecules in a specific location within the target cell, such as at the interior of the target cell membrane, or preferentially at a specific location or range of locations within the target cell, for example associated with an organelle or interior structure of the target cell, such as the nuclear or mitochondrial membrane. The particles may be adapted to enter the target cell, for example by endocytosis following targeting to a target molecule on the target cell membrane, while particles are not taken up, or are taken up to a lesser extent, by non-target cells.

Typically the size of a particle adapted to enter the cell is in the range of 20 nm to 2 um, optionally to 5 um. In contrast, particles adapted to associate with the exterior of the membrane may be adapted so as not to promote its uptake by endocytosis and may have a coating that does not promote endocytosis, or acts to delay or hinder it. Typically the size of a particle destined for the exterior of the cell is in the range of 20 nm to 5 um.

In a particularly preferred embodiment of the method a first particle is provided that is adapted to enter a target cell and a second particle is provided that is adapted to associate with the exterior of the cell membrane. The field enhancement of a pair of particles in proximity is very much greater than that of a single particle alone and, for particles that have higher permittivity or conductivity than the surrounding environment, is greatest in the region between the particles. In this way a particle arrangement is provided in which the enhanced field lies across the cell membrane and so leads to a large reduction in the threshold field for electroporation. One or both of the first and the second particle may be targeted to the target cell as described herein.

According to another aspect the invention relates to an apparatus for diagnosis or treatment of a disease condition in a subject using particles or nanoparticles and time-varying electromagnetic or electric fields, characterised in that a means is provided to associate a particle with the membrane of a target cell, a diagnostic or therapeutic species is provided to the external environment of the cell, and a means is provided for exposing the target cell to an electric field sufficient to cause reversible electroporation allowing the species to enter the cell.

The species may comprise a drug, a diagnostic marker, a particle or a nucleic acid construct comprising DNA or RNA, for example for gene therapy or application of gene silencing by means of RNA interference. The species may comprise a particle such as a marker particle such as a quantum dot or a particle loaded with a further species such as are listed above.

According to another aspect the invention relates to an apparatus for treatment of a disease condition in a subject using particles or nanoparticles and time-varying electromagnetic or electric fields, characterised in that a means is provided to introduce a particle to the exterior of a target cell and to allow the particle to bind to a target molecule on the cell membrane, and a means is provided for exposing the target cell to an electric field sufficient to cause cell death by non-thermal means, for example irreversible electroporation of the cell. It is understood that in some embodiments cells may undergo apoptosis as a result of exposure to the enhanced electric field, and that electroporation may be the cause of cell death, without immediate lysis of the cell following the irreversible electroporation procedure.

Herein the term subject is meant to include a living organism including individual humans and animals. The terms subject, host organism and target organism are used interchangeably.

The apparatus of the invention may be used in order to treat for example neoplasia, cancerous cells, or to treat infections caused by fungi, vira, bacteria or other microorganisms.

Ideally the means to introduce particles to the target cell includes a particle delivery device that administers particles to the target cells or a region of tissue comprising them, for example by topical or systemic administration. Particles so delivered are preferably adapted to associate with or to enter the target cell as described herein.

Means may be provided as part of the apparatus to provide a first particle type, adapted as described above to enter a target cell, and a second particle type, adapted to bind to a target molecule on the exterior of a target cell membrane. In a preferred embodiment, the first particle type is also targeted to a target cell, as described above, so that it enters a target cell preferentially. In addition entry of the first particle type into non-target cells may be minimised.

Particles may be administered systemically by, for example, administering into a body fluid such as blood, lymph, cerebrospinal fluid, so that the fluid acts to carry the particles to the target cells. In some embodiments the target cells may be within the body fluid into which the particles are administered. In some embodiments the body fluid may act as a transfer medium to carry, or allow transmission of, the particles to the target cells. The target cells may be bacteria, spores, vira or mammalian cells, for example leukaemic or virally infected cells within the body fluid. The target cells may instead be localised, for example in a local seat of infection, a region of neoplasia or a tumour. Particles may be administered topically, i.e. locally to the region of the target cells.

Particles may be administered by any means known in the art, such as injection and/or infusion and/or electroporation through skin and/or inhalation and/or absorption through mucal membranes and/or via the digestive tract. Devices for administration include: a syringe, cannula, catheter, inhaler, implanted release device, capsule or ingestible preparation.

In preferred embodiments of the invention there is provided a means to expose the target cells to a variable electric or electromagnetic field comprising: at least a first and a second electrode and a control means for applying a variable potential to the first and second electrodes, whereby target cells within the electric field are electroporated and non-target cells are not. The field strength may be chosen according to the nature of the disease condition, the nature of the particles, and the proportions of target and non-target cells that are to be electroporated on average in a given treatment, and whether irreversible or reversible electroporation is desired.

In a preferred embodiment the apparatus comprises a device for providing a time varying first electric potential to the first electrode and second electric potential to the second electrode, and a programmable unit adapted to control the device in response to instructions stored on a storage medium accessible by the programmable unit.

A preferred embodiment the method of the invention comprises a process in which an applied field strength is selected, the target cells are electroporated, and a diagnostic process is used to assess the degree and effect of the electroporation. The field parameters are then adjusted and the process repeated. In a process in which target cells are electroporated within a body of tissue, for example within a patient being treated for disease, the parameters required will be determined partly by the location of the cells within the body of the subject and the configuration of the electrodes. Therefore once the parameters required for effective electroporation have been determined, those same parameters may be used in subsequent treatments. Therefore according to an aspect of the invention the method comprises a step of establishing the required potentials and time profile of potentials to be applied to the electrodes set out as above sufficient to achieve the desired effect of electroporation, and using those parameters in subsequent use of the method.

In a further preferred embodiment, the invention comprises a process as above wherein the field parameters are selected so as to cause death of target cells, for example by irreversible electroporation, and in preferred embodiments the parameters are selected so as to cause death of target cells and to cause lesser degrees of, or essentially no, damage to non-target cells.

In a preferred embodiment the apparatus comprises: at least a first and second electrode adapted to be located externally to the body of a subject or to be placed in contact with the skin of the subject. It is an advantage of the present invention that the field required for electroporation or to cause death of target cells is sufficiently low that in contrast with the prior art electrodes may be used in some embodiments that do not need to be implanted.

In some embodiments the apparatus may comprise one or more electrodes that are adapted to be implanted within the body of the subject. Suitable electrodes are known in the art as used for conventional electroporation and for irreversible electroporation treatment. In preferred embodiments the electrodes are adapted to provide a field localised in the vicinity of a region of target cells, such as for example a seat of infection or a tumour. In an alternative embodiment the electrodes are adapted to provide a field over a larger region, so as for example to treat a delocalized condition over a region of the subject's body.

In some embodiments the apparatus comprises means to change the orientation of the applied field during a course of treatment. In some embodiments the apparatus includes means to move one or more electrodes with respect to the subject or vice versa.

According to a further aspect of the invention there is provided a method of targeted electroporation of a target cell comprising the steps of: causing a particle of high permittivity or high conductivity to associate with the exterior of a target cell membrane and exposing the target cell to an electric field sufficient to cause electroporation of the cell.

In preferred embodiments the particle is a dielectric particle. Preferably the particle has a high permittivity with respect to the cell and a surrounding medium.

In some embodiments the particle has a high conductivity with respect to the cell and a surrounding medium, and comprises a conductive material or comprises a conductive core, such as a metal, for example gold, or a metal oxide, for example Fe₃O₄.

Particles are preferably of higher permittivity or conductivity than the mean permittivity or conductivity of the composite medium environment in a region surrounding them. Typically that region comprises one or more of the extracellular fluid; extracellular matrix and associated proteins; cell membranes of the target cell and surrounding cells; cell surface molecules such as membrane proteins, glycoproteins and sugars. The permittivity or conductivity of the surrounding environment is therefore a composite permittivity or conductivity derived from the presence and parameters of various composite components, and therefore may take a range of values.

In a preferred embodiment the relative permittivity of the particle or a material of which the particle is substantially formed is greater than that of physiological saline solution or blood, more preferably greater than 88.

As a result of selecting the particle type to have a high conductivity or permittivity; its location with respect to the cell; and the specific field characteristics, in preferred embodiments the target cell experiences reversible pore formation in the cell membrane at reduced applied field strength, thus reducing the risk of damage to the target cell and substantially preventing electroporation of neighbouring non-target cells.

In alternative preferred embodiments the field characteristics are selected such that the target cell experiences irreversible pore formation in the cell membrane at reduced applied field strength, so causing death of the target cells and substantially preventing death of neighbouring non-target cells.

According to a further aspect, the invention relates to an apparatus for electroporation of a targeted cell, comprising: means to provide particles to a target cell, the particles being adapted to associate with the target cell, and means to apply an electric field to a target cell, comprising a first and a second electrode and a device to apply time-varying potentials to the electrodes.

In a preferred embodiment the apparatus is adapted for irreversible electroporation of a targeted cell, the electric field being sufficient to cause irreversible electroporation in the cell membrane when enhanced by the presence of the particles.

Electrodes and particles may have characteristics as described above. Further characteristics of electrodes and particles that are used in various embodiments of the invention are set out below.

In a preferred embodiment the apparatus comprises a device for providing a time varying first electric potential to the first electrode and second electric potential to the second electrode, and a programmable unit adapted to control the device in response to instructions stored on a storage medium accessible by the programmable unit.

According to a further aspect of the invention there is provided a method for treatment of a disease in a subject by means of electroporation of target cells within the subject, comprising the steps of:
a) administering particles to the subject, either systemically or topically in the region of the target cells, the particles being adapted to associate with the membrane of the target cells;
b) applying an electric field to a region of the subject within which one or more target cells are located sufficient to cause electroporation of the targeted cells.

In a first embodiment as above the applied field is selected to cause irreversible electroporation and death of the target cells.

In an alternative embodiment as above, the applied field is selected to cause reversible electroporation, the method further comprising the step of:
c) before applying the electric field, providing a species to be taken up by the target cell to the vicinity of the target cell

Preferably the method includes the step of allowing a chosen time interval to elapse between step (a) and step (b) to allow time for at least one particle to become associated with the target cell before the field is applied.

Optionally particles may be located and/or tracked within the body fluid or the site of action by means known in the art, for example MRI, ultrasound or computer tomographic (CT) scanner, chosen according to the nature of the particles in use. In addition the electric field may be applied at a chosen point depending on the results from the location and tracking process, and associated processing and display equipment may be provided in order to enable this.

In a further embodiment the method comprises the steps of:
a) administering particles to the subject, either systemically or topically in the region of the target cells, the particles being adapted to enter the cytoplasm of the target cells;
b) applying an electric field to a region of the subject within which one or more target cells are located sufficient to cause electroporation of the targeted cells.

In a first embodiment as above the applied field is selected to cause irreversible electroporation and death of the target cells.

In an alternative embodiment as above the applied field is selected to cause reversible electroporation, the method further comprising the step of:
c) before applying the electric field, providing a species to be taken up by the target cell to the vicinity of the target cell

Preferably the method includes the step of allowing a chosen time interval to elapse between step (a) and step (b) to allow time for at least one particle to enter at least one target cell.

In a preferred embodiment the method comprises the steps of:
a) administering a first particle type to the subject, either systemically or topically in the region of the target cells,
b) administering a second particle type to the subject, either systemically or topically in the region of the target cells,
c) allowing at least one particle of the first particle type and at least one particle of the second particle type to become associated with one or more target cells,
d) applying an electric field to a region of the subject within which one or more target cells are located sufficient to cause electroporation of the targeted cells.

Preferably the first particle type is adapted to enter the cytoplasm of the target cells and the second particle type is adapted to associate with a target molecule on the exterior of the membrane of the target cells.

In a first embodiment as above the applied field is selected to cause irreversible electroporation and death of the target cells.

In an alternative embodiment as above the applied field is selected to cause reversible electroporation, the method further comprising the step of:
e) before applying the electric field, providing a species to be taken up by the target cell to the vicinity of the target cell

Preferably the method includes the step of allowing a chosen time interval to elapse between step (a) and step (b) to allow time for particles of the first type to enter the target cells and the step of allowing a chosen time interval to elapse between step (b) and step (d) to allow time for particles of the second type to associate with target molecules on the exterior of the target cells before the field is applied.

In preferred embodiments the particles become associated with the target cells so as to form an arrangement of particles in which the particles act together to enhance the electric field in their vicinity, so reducing the applied electric field required for electroporation of the target cell. Such enhancement may take place when both particles are on the same side of the cell membrane. In the preferred embodiment in which the first particle type is adapted to enter the target cell and the second particle type is adapted to be bound to target molecules on the exterior of the cell, the first and second particles act together to enhance the electric field across the portion of the cell membrane that lies between the particles, causing a pore to form in that region when a suitable field is applied.

In preferred embodiments a delay is provided between the administration of the first and the second particle types. The delay is typically in excess of 10 minutes.

In preferred embodiments a delay is provided between administration of the second particle type and application of the electric field. The delay is typically in excess of several minutes, ideally more than 10 minutes.

According to a further aspect the invention relates to an apparatus and a method for treatment of a disease condition in a subject using particles or nanoparticles and time-varying electromagnetic or electric fields, characterised in that a means is provided to associate a particle selectively with a target cell in a liquid medium and a means is provided for exposing the target cell to an electric field sufficient to cause electroporation of the cell.

In a first embodiment treatment of a disease in a subject by causing death of target cells located at least partially in a body fluid comprises the steps of: administering particles to the body fluid, allowing the particles to become associated with target cells within the body fluid, applying an electric or electromagnetic field to the target cells and particles within the body fluid, thereby causing cell death by primarily non-thermal means, for example by irreversible electroporation of the target cell membrane.

In a further embodiment treatment of a disease in a subject by causing electroporation of target cells located at least partially in a body fluid comprises the steps of: administering particles to the body fluid, allowing the particles to become associated with target cells within the body fluid, providing a species to be introduced into the cells to the vicinity of the cells within the body fluid, and applying an electric or electromagnetic field to the target cells and particles within the body fluid, thereby causing the species to be introduced into the target cells.

An example of a body fluid may be blood or a blood component such as plasma, cerebrospinal fluid, or bone marrow. Optionally treatment of the body fluid is performed on the fluid after it has been removed from the human or animal body or is performed whilst the fluid is outside the human or animal body.

In a preferred embodiment an apparatus comprises particles adapted to bind to a target molecule on the surface of a target cell, or to enter a target cell, as described previously; means to administer particles into the body fluid; optionally, means to provide a species to be introduced into the cells within the body fluid and at least a first and a second electrode adapted to apply an electric field to a region of body fluid containing target cells and a device for providing time varying potentials to the first and the second electrodes.

Preferably the electrodes apply an electric field to a region through which a body fluid flows, so bringing target cells into the region with the flow. In an embodiment the electrodes are adapted to apply a field to a perfused region such as a blood vessel in vivo, for example a blood vessel near the surface of the body.

In an alternative embodiment the apparatus includes means for applying an electric field to a body fluid externally to the body. Preferably the apparatus comprises an extracorporeal flow system comprising a flow cell through which the body fluid, such as blood, may flow, the flow cell being adapted to apply an electric field to the fluid. The flow system may then return the treated fluid to the subject.

A preferred embodiment of the method according to this aspect of the invention includes the steps of: administering particles systemically to the subject, providing a species to be introduced into the target cells within the body fluid, and then providing an electric field in a region containing the body fluid or through which the body fluid may flow. The method preferably includes a repeated application of the field sufficient to cause electroporation of the target cells as they move into a region where the electric field is applied.

The method envisages that target cells may be delocalized within the body fluid of the subject, and present at low concentrations within the body fluid, so may be electroporated gradually by repeated applications of the field over time. It is a feature of the invention that the reduction in field strength and electrode potentials needed to achieve electroporation facilitates repeated applications of the field compared with prior art apparatus and methods.

According to a further aspect of the invention there is provided a method for causing electroporation of a target cell, comprising the steps of: providing a particle arrangement of at least a first and a second particle in proximity to the target cell; applying an electric or electromagnetic field to the target cell and particle arrangement, the first and the second particles being arranged such that they cause enhancement of the component of the field appearing across a region of the target cell in the vicinity of the first and second particles, so causing electroporation of the target cell. In a preferred embodiment the enhanced field causes cell death; in an alternative embodiment the enhanced field may cause reversible electroporation.

In a preferred embodiment the first and the second particle are arranged so that a region or component of the target cell lies between them.

Preferably the first and the second particle are located on opposite sides of the target cell membrane, or in some embodiments the cell nucleus or other organelle within the target cell in the direction of a component of the applied electric field, thereby enhancing the applied field appearing across the cell, organelle or component. In one embodiment a field may then be applied so as to cause irreversible electroporation of the cell membrane or nuclear membrane, leading to cell death. In an alternative embodiment a field may then be applied so as to cause reversible electroporation of the cell membrane or nuclear membrane, allowing a species to enter the cell or the nucleus.

In a preferred embodiment sufficient particles are provided such that on average a proportion of target cells have a particle arrangement as above. The number of particles supplied and the applied field strength may be chosen to optimize the proportion of target and non-target cells killed or electroporated by the field.

The electric field may be applied in a first direction and then subsequently in a different orientation, for example by moving one or more electrodes used to provide the field relative to the cell. As described further below, a plurality of electrodes may be provided to allow the field direction to be chosen or varied during the treatment.

A further aspect of the invention provides a method for selectively electroporating a cell, the method comprising the steps of: providing a particle arrangement of at least a first and a second dielectric particle, where the first dielectric particle is within the cytoplasm of the cell and the second dielectric particle is on the extracellular side of cell.

An alternative approach is where both the first and second dielectric particle are at the extracellular side of the cell or both particles are in the cytoplasm of the cell. However, both alternatives are likely to give lesser effect than the first mentioned arrangement.

Ideally a coating is provided on at least one of the particles so as to render it specific for the target cell type in such a way that at least one particle binds selectively to one or more target molecules in the cellular membrane.

Additionally the particles are exposed to an alternating electric field, the alternating electric field being provided by at least the first and the second electrodes and being of sufficient frequency and amplitude so as to cause a concentration of field flux between the at least two particles so as to cause electroporation of the membrane of the target cell.

The field amplitude and frequency can be chosen so that irreversible pore formation is achieved in a cell membrane in the vicinity of the two dielectric particles and so that adjacent cells are unaffected, because it is only the field flux concentration between the two dielectric particles that is strong enough to mediate molecular disarrangement in the cellular membrane.

In alternative embodiments the field amplitude and frequency can be chosen so that reversible pore formation is achieved in the vicinity of the two dielectric particles and so that adjacent cells are unaffected.

Preferred embodiments of this and other aspects of the present invention may comprise some or all of the following features. Unless otherwise indicated, in the following the term ‘electroporation’ may encompass both reversible and irreversible electroporation. According to the embodiment, the applied field parameters are selected to cause either irreversible electroporation, leading to cell death, or reversible electroporation.

In preferred embodiments of the invention, the method may further include the step of performing an analysis of the extent of electroporation, said step comprising extracting biological material from the host organism and subjecting extracted material to an investigation in order to determine the degree to which the species to be introduced to the cells has in fact been introduced. Alternatively, the analysis may involve a non-invasive technique such as ultrasonic investigation, computer tomography (CT), X-ray or magnetic resonance image analysis.

In the present context the term “target cell” is related to a biological form of life comprising for example, a microorganism, a virus, or an eukaryote cell.

In a preferred embodiment of the invention, the microorganism is hosted inside a mammalian cell which is serving as a reservoir for the infection.

In a preferred embodiment of the invention, the microorganism is resistant to common chemotherapies such as anti-biotics such as but not limited to methicillin-resistant Staphylococcus aureus (MRSA), vancomycin-resistant Staphylococcus aureus (VRSA), penicillin resistant Streptococcus, anti-biotic resistant strains of Mycobacterium tuberculosis, penicillin resistant Enterococcus, multi-drug resistant Pseudomonas aeruginosa, clindamycin (or fluoroquinolone) resistant Clostridium difficile (diarrheal disease) and multi-drug resistant Escherichia coli.

In an embodiment of the invention, the first and a second electrode are separated by a distance being at the most 1 m, preferably being at the most 0.9 m, such as at most 0.8 m, 0.7 m, 0.6 m, 0.5 m, 0.4 m, 0.3 m, 0.2 m, 0.1 m, or at most 0.05 m, more preferably being at the most 0.04 m, and even more preferably at most 0.03 m such as at most 0.02 m, such as at most 0.01 cm.

For example the first and the second electrode may be separated by a distance in the range of 0.01-1 m, such as in the range of 0.01-0.05 m, 0.05-0.1 m, 0.1-0.2 m, 0.2-0.3 m, 0.3-0.4 m, 0.4-0.5 m, 0.5-0.6 m, 0.6-0.7 m, 0.7-0.8 m, 0.8-0.9 m or such as in the range of 0.9-1.0 mm.

Typically, the first and the second electrode are separated by a distance, which is at least 0.01 m such as at least 0.03 m or 0.05 m.

In a preferred embodiment related to electroporation of target cells present within an organism, at least a part of the target cells in the organism is positioned between the first and the second electrode. For example, at least 1% of the target cells are positioned between the first and the second electrode, such as at least 5, 10, 20, 30, 40, 50, 60, 70, 80, 90, 95, 97.5, 99, 99.5, 99.9 or 100% of the 1% of target cells are destroyed by the electrical field imposed by the first and the second electrode.

Preferably, in use, the target cells located in the organism are positioned between at least the first and the second electrode as part of the method. The first or the second electrode may be attached directly to surface of the organism.

According to the embodiment, the first and/or the second electrode may have different shapes or dimensions. For example, the first and/or the second electrode may have a substantial form chosen from the group of a sheet, a plate, a disc, a wire, a rod; or any combination thereof.

In a preferred embodiment of the present invention, the first and the second electrode may for example be a combination of a point electrode and a sheet electrode.

In a preferred embodiment of the present invention the first electrode and the second electrode are facing each other. For example, they may be positioned at opposite sides of an organism hosting the target cells creating a field that is optimal for concentrating the electric field flux in the vicinity of the particle or two-particle arrangement.

The first and second electrodes may take any appropriate form as described in the art. The present invention offers the advantage that the field needed for successful electroporation of target cells is lower than in the prior art, so allowing a wider range of electrode types and locations during treatment to be used.

In a preferred embodiment relating to treatment of disease in a subject one or both of the first and second electrodes are implanted within the body of the subject (i.e. the organism hosting the target cells). One or both of the first and second electrodes may be implanted in the vicinity of a group of target cells, such as a tumour.

An implanted electrode may be positioned within, adjacent to or around the group of target cells. In an embodiment one or both of the first and second electrode may take the form of a probe that may be manipulated by a clinician to apply an electric field locally to the probe, so allowing the clinician to position the electrode in order to apply the field to a chosen region. The probe may be adaptable to be used during a surgical procedure that exposes a deep-seated group of target cells, for example a tumour, the probe then being applied to a chosen area by the clinician.

The first and second electrodes may both take the form of probes, and both might be usable in this way. Alternatively the second electrode may be adapted to remain in a fixed location while the first is moved. In some embodiments the second electrode may have an extended conducting surface, for example in contact with soft tissue of the subject, and may in some embodiments be in contact with the skin of the subject.

In some embodiments the electric field may be applied in a single orientation between the first and second electrodes. In further embodiments the electric field may be applied in further orientations relative to the target cells or to an organism that hosts the target cells, such as in the case of treatment of disease in a subject. This may be achieved in some embodiments by changing the disposition of one or more electrodes relative to each other or to the subject, for example by moving either one or more electrodes or moving the subject. In further embodiments more than two electrodes may be provided in order to allow the field to be applied in a number of different orientations.

An electrode, for example a first electrode in a pair of electrodes, may be formed from a variety of different materials. Optionally the first electrode and a second electrode are formed from the same material. Typically, the electrodes are formed from metals or alloys. The first and the second electrode may for example comprise a metal selected from the group comprising: silver, gold, platinum, copper, carbon, iron, graphite, chromium, nickel, cobalt, titanium, mercury or an alloy thereof.

It is also envisaged that an electrode may comprise a conducting liquid, for example saline solution or a conductive gel, and may essentially consist of a conducting liquid.

In preferred embodiments, a typical dimension of the particles is less than 0.1 micrometre (μm).

The core of embedded particles can be formed from a range of materials capable of behaving as a dielectric such as but not excluded to carbon, iron oxide (various forms), titanium dioxide, cerium oxide or silicon dioxide.

In other preferred embodiments the particles may be in the form of nanoparticles which may take the specific form of nanotubes. Alternatively the particles may be substantially spherical, for example cubic or octahedral, or they may be nanorods.

The dimension or/and structure of electrodes typically depends on the dimension of an optimal target area of a host organism within which the target cells are located.

Advantageously the length and width of the electrodes are of the same order of magnitude as the radius of the target area.

The electrodes can be formed by as little as a coating of a few atom layers of conductive material.

In an embodiment a first and/or second electrode has a thickness in the range of 0.001 μm-2000 μm, such as 0.001 μm-1 μm, 1 μm-20 μm, 20 μm-200 μm, and 200 μm-2000 μm.

In a preferred embodiment a liquid sample, in which cells are supported, is exposed to an alternating electric field provided by the first and the second electrode. It is important that the alternating electric field has a sufficient frequency and sufficient amplitude and is applied for a sufficient duration of time to cause reversible electroporation of the target cell whereas non-target cells remain relatively unaffected.

The term alternating electric field relates to electric fields that change over time. The alternating electric field may e.g. be the electric field that occurs from periodically shifting the polarity of two electrodes between positive/negative and negative/positive, that is connecting an AC source to the electrodes. Also, the alternating electric field may comprise one or more DC pulses.

Pulses may have a duration in the range 1 ns to 100 ms, preferably in the range 10 ns to 1 ms. Pulses may generally have durations and repetition rates, patterns and numbers of pulses as known in the practice of electroporation, the use of particles in accordance with the invention increasing the effect of each pulse or train of pulses.

In a preferred embodiment of the invention, the frequency of the alternating electric field is at the least 10 kHz, preferably being at least 50 kHz, and more preferably being at least 100 kHz.

In another preferred embodiment of the invention, the frequency of the alternating electric field is at the least 100 kHz, preferably being at least 500 kHz, and more preferably being at least 1000 kHz.

In another preferred embodiment of the invention, the frequency of the alternating electric field is at the least 1 MHz, preferably being at least 50 MHz, and more preferably being at least 100 MHz.

In another preferred embodiment of the invention, the frequency of the alternating electric field is at the least 100 MHz, preferably being at least 500 MHz, and more preferably being at least 1 GHz.

In another preferred embodiment of the invention, the frequency of the alternating electric field is at the least 1 GHz, preferably being at least 500 GHz, and more preferably being at least 1 THz.

For example, the frequency of the alternating electric field may be at least 10 kHz, such as at least 30 KHz, 100 KHz, 300 KHz, 1 MHz, 10 MHz, 30 MHz, 100 MHz, 300 MHz, 1 GHz, 10 GHz, 30 GHz, 100 GHz, 300 GHz, such as at least 1000 GHz.

Preferably the frequency of the alternating electric field is at most 500 GHz, such as at most 1000 GHz.

The amplitude of the alternating electric field, that is, the maximum potential difference between the first and the second electrode, is typically at most 100KV, such as at most 30KV, 10KV, 1KV, 300V, 100V, 30V, 10V such as at most 1 V.

In an embodiment directed to irreversible electroporation, the maximum field applied between the electrodes is preferably selected to be below 1500V/cm, more preferably in the range 500 to 1000V/cm, more preferably still in the range 100 to 500V/cm, and in some embodiments 10 to 100V/cm.

In an embodiment directed to reversible electroporation, the maximum field applied between the electrodes is preferably selected to be in the range 100 to 500V/cm, more preferably in the range 20 to 100V/cm, more preferably still in the range 5 to 50V/cm, and in some embodiments 1 to 5 V/cm.

The electroporation, and in some embodiments, destruction of the said target cells in the host organism is strongly dependent on the design of, and the distance between, the first and the second electrode, the electrode structure and the materials of at least first and second particles located within and in proximity of the extracellular side of the cell membrane or cell wall (bacteria) and the potentials and frequencies applied to the first and the second electrode.

In a highly preferred embodiment of the invention, the first potential of the first electrode and the second potential of the second electrode, and thus the alternating electric field between the first and the second electrode, are modulated so as to yield electroporation of target cells in a target area of least 30% of the target cells, such that at least 40% of the target cells, preferably of at least 50% of the target cells, and more preferably of at least 60% of the target cells, such as of at least 70%, 80%, 90%, 95%, 97.5%, 99%, 99.5% or 99.9% such as approximately of 100% of the target cells are electroporated.

In another highly preferred embodiment of the invention, the first potential of the first electrode and the second potential of the second electrode, and thus the alternating electric field between the first and the second electrode, are modulated so as to yield a specific reversible electroporation or destruction of target cells in a target area and lack of electroporation or destruction of at least 70% of the non-target cells, such that at least 75% of the non-target cells, preferably of at least 80% of the non-target cells, and more preferably of at least 85% of the non-target cells, such as of at least 90%, 95%, 97.5%, 99%, 99.5% or 99.9% such as approximately of 100% of the non-target cells.

In another highly preferred embodiment of the invention, the first particle and the second particle can mediate a field flux concentration compared to of the potential supplied by the first and the second electrode, so that the field flux concentration between said particles, existing in an area between the two particles, is at the least a factor 1.1, preferably being at least a factor 10, and more preferably being at least a factor 100 compared to the surrounding field flux.

In another highly preferred embodiment of the invention, the first particle and the second particle are loaded in the extracellular fluid of the target organism e.g. for humans that means the blood stream, in a time separated sequential manner so that the first particle has time to accumulate in the cytosol of the target cells before the second particle is loaded for binding to the extracellular side of the cellular membrane or cell wall (bacteria).

In an embodiment of the invention, the loading in the extracellular fluid of the first and a second particles, are separated in time by an interval being at the most 30 days, preferably being at the most 20 days, such as at most 10 days, 9 days, 8 days, 7 days, 6 days, 5 days, 4 days, 3 days, 2 days, 1 day, 18 hours, 16 hours, 14 hours, 12 hours, 10 hours, 8 hours, 6 hours, 4 hours, or at most 2 hours, more preferably being at the most 1 hour, and even more preferably at most 30 minute such as at most 20 minute, such as at most 10 minutes.

Typically, the alternating electric field is provided by modulating the polarity of the two electrodes.

The alternating electric field may have a substantial form chosen from the group consisting of: rectangular, sinusoidal, saw-tooth, asymmetrical triangular, symmetric triangular; or any combination thereof.

Also, the alternating electric field, in the frequency domain, may comprise at least a first and a second frequency component.

In an embodiment of the invention, the duration for which the alternating electric field is applied is at most 3600 seconds, such as at most 3000, 2000, 1000, 500, 250, 100, 50, 40, 30, 20, 10, 5, 4, or 3 seconds, such as at most 1 second.

For example, the duration for which the alternating electric field is applied is in the range of 0.01-3600 seconds, such as in the range of 0.1-1, 1-5, 5-10, 10-25, 25-50, 50-100, 100-250, 250-500, 500-1000, or 1000-2000 seconds, such as in the range of 2000-3600 seconds. In a preferred embodiment of the invention, the duration for which the alternating electric field is applied is in the range of 5-100 seconds, such as 6-90 seconds, 7-80 seconds, 8-70 seconds, 9-60 seconds and 10-50 seconds.

In a preferred embodiment of the invention, the duration for which the alternating electric field is applied is at most 250 second, preferably for at most 100 second such as for at most 30 seconds.

The particles are preferably coated with a composition that will enhance the binding to target cells over non-target cells in the host organism. In preferred embodiments the coating comprises at least one molecular component that increases the affinity of the particle for a target cell membrane, organelle, lipid, carbohydrate, a nucleic acid (such as chromosomal DNA and/or plasmid DNA and/or any type of RNA), a protein (e.g., from the group comprising enzymes, structural proteins, transport proteins, ion channels, toxins, hormones, and receptors) or small molecule that can be bound to the cellular membrane either in form of an agonist and/or antagonist compared to its affinity for non-target cells.

Another aspect of the invention relates to a device for selective electroporation of target cells in an organism, the device comprising: a set of electrodes to be functionally associated with the device, an electrical interface between the device and the electrode arrangement for applying an alternating electric field between the electrodes, an electronic circuit capable of providing a fast switching high amplitude signal and a programmable unit.

The programmable unit ideally contains instructions, preferably computer readable such as software, adapted to facilitate controlling, monitoring, and/or manipulating of the device prior to operation, under operation, and/or after operation.

The programmable unit preferably comprises at least one computer having one or more computer programs stored within data storage means associated therewith, the computer system being adapted to control a system employing first and second electrodes arranged to apply an alternating electric field.

The programmable unit may in the context of the present invention be chosen from the non-exhaustive group of: a general purpose computer, a personal computer (PC), a programmable logic control (PLC) unit, a soft programmable logic control (soft-PLC) unit, a hard programmable logic control (hard-PLC) unit, an industrial personal computer, or a dedicated microprocessor.

The present invention also relates to a computer program product, such as one recorded on a data storage medium, being adapted to enable a computer system, comprising at least one computer having data storage means associated therewith to control, monitor, and/or manipulate the device prior to operation, under operation, and/or after operation.

Advantageously a computer readable medium has stored thereon a set of routines for enabling a computer system comprising at least one computer having data storage means associated therewith to control, monitor, and/or manipulate the device prior to operation, under operation, and/or after operation. The programmable unit is ideally capable of: checking that electrodes are functionally associated with the device, providing a voltage protocol to the electrodes, and setting total time, amplitude and frequency of the applied signal. Secondly being able to repeat the voltage protocol in a number of series with a given time interval between each exposure.

Optionally target cells are exposed whilst in a host organism to an alternating electric field via the electrode arrangement, said alternating electric field being provided by the first and the second electrode and having a sufficient frequency and a sufficient amplitude so as to cause the selective reversible electroporation or irreversible electroporation, according to the embodiment, of the target cells in the host organism, and optionally performing an analysis on the exposed organism which part comprises an analysis of the degree to which a species has been introduced to target and non-target cells in the affected area of the host organism.

The device may further comprise an electrical power supply for supplying the high voltage needed for the first and second electrodes to achieve the needed effect on the said first and second particle.

In an embodiment of the invention, the programmable unit comprising the software furthermore ensures that the device checks that electrodes are functionally associated with the device.

According to a further aspect the invention provides a composition comprising: a plurality of particles adapted for use in a method and system for causing the electroporation of a target cell, the particles being adapted to associate with the target cells and adapted to cause an enhancement of an applied electric or electromagnetic field in their vicinity.

According to a further aspect the invention provides a composition comprising: a plurality of particles adapted for use in a method and system for causing the death of a target cell by primarily non-thermal means, for example by irreversible electroporation, the particles being adapted to associate with the target cells and adapted to cause an enhancement of an applied electric or electromagnetic field in their vicinity.

In various embodiments the particles have properties as described herein. The composition of the invention may comprise further components to maintain the efficacy and useful life of the composition, for example to maintain the particles in suspension, to aid their administration to a subject, to aid their circulation within the body fluid of a subject for example in the blood, or to aid their absorption into the soft tissue of a subject.

According to a further aspect of the invention a system is provided for causing electroporation of a target cell comprising: at least a first composition as described herein, comprising a plurality of at least a first particle type, the particle being adapted to associate with the target cell and adapted to cause an enhancement of an applied electric or electromagnetic field in its vicinity; and an apparatus for applying an electric field to one or more target cells and to one or more particles associated with the target cells, comprising at least a first and a second electrode and a device for providing a first electric potential to the first electrode and a second electric potential to the second electrode, the device comprising a programmable unit adapted to control the device in response to instructions associated with the programmable unit; and a method as disclosed herein, carried out using the composition and the apparatus.

In a first embodiment the system comprises a device adapted to provide first and second electrode potentials and their time course selected to achieve irreversible electroporation of the target cells.

In an alternative embodiment directed to reversible electroporation the system further comprises a means to provide a species to be introduced into the target cell to the vicinity of the target cells.

In a preferred embodiment the system may additionally comprise: a second composition comprising at least a second particle type, the second particle type being adapted to associate with the target cell and adapted to cause an enhancement of an applied electric or electromagnetic field in its vicinity. The second particle type may have any of the properties as described herein.

In various embodiments the particles have properties as described herein. In preferred embodiments at least one particle type comprises a coating that makes it selective for the target cell type in such a way that at least one particle will bind selectively to one or more target molecules in the cellular membrane.

According to a further aspect of the invention a system is provided for treating a disease in a subject comprising the composition, apparatus and method as described above.

In a further embodiment the system additionally comprises: means for administering the composition to the subject, the administration being topical i.e. in the vicinity of the target cells, systemic, or both, said means being for example (but not being limited to): a syringe, a cannula, a catheter, an inhaler, an implanted release device, a capsule or ingestible preparation or means for administration using electroporation.

A first means for administration may be used with the first composition and a second means for administration may be used with a second composition.

According to a further aspect of the invention there is provided an apparatus and a method for a biological process, the method comprising the steps of: providing a number of at least a first particle type to cells in culture, the cells comprising target cells, the particles adapted to associate selectively with target cells; allowing the particles either to bind to target molecules on the surface of the target cells or to be taken up inside the target cells; and applying an electric field to the cells in culture, so causing electroporation of target cells.

In a first embodiment the electric field is selected so as to cause irreversible electroporation and cell death.

In a further embodiment the electric field is selected so as to cause reversible electroporation and the method comprises the further step of providing a source of a species to be introduced into the target cells and introducing the species to a medium contacting the cells. In a preferred embodiment the target cells are in contact with a liquid media, the particles are mixed with the media and the field is provided within the media by electrodes either within the media or external to it. The electrodes may be insulated from the media. There may be an air gap between the electrode surface and the media.

Preferably the method comprises provision of first and second particle types as described previously.

In a preferred embodiment the apparatus may comprise: particles of at least a first particle type, and optionally particles of a second particle type; a culture container adapted to allow the provision of an electric field to the cells in culture, and at least a first and a second electrode adapted to provide a field to the culture container. In an alternative embodiment, the apparatus may comprise a flow system having a flow cell through which cells may flow in a liquid medium, the flow cell being adapted to provide an electric field to the flowing cells, such that target cells are electroporated selectively.

According to a further aspect of the invention there is provided an apparatus and a method for an analytical process, the method comprising the steps of: providing a plurality of a first particle type to cells in a liquid sample; allowing the particles either to bind to target molecules on the surface of the cells or to be taken up inside the cells; providing a species to be introduced into the cells within the liquid sample, and applying an electric field to the liquid sample, so causing a species to be introduced into the target cell by means of electroporation.

According to a further aspect of the invention there is provided an apparatus and a method for an analytical process, the method comprising the steps of: providing a plurality of a first particle type to cells in a liquid sample; allowing the particles either to bind to target molecules on the surface of the cells or to be taken up inside the cells; and applying an electric field to the liquid sample, so causing lysis of cells by irreversible electroporation of the cell membrane.

This aspect relates to analytical or diagnostic processes in which it is desired to release cell contents into a liquid sample, for example in analysis of DNA, RNA, proteins or other constituents of the cell. The method of the invention provides a ready means for lysis of cells at low electric fields and hence with lower electrode potentials than in the prior art. The particles may be taken up into the cells or be associated with the external surface of the cell membrane.

In a preferred embodiments the particles are selective for target cells by binding selectively to target molecules on the target cell membrane as described previously, so enabling selective reversible electroporation of target cells while non-target cells remain unaffected. In a preferred embodiment a first particle type is taken into the cell and a second particle type is bound to the exterior of the cell membrane, so reducing the threshold field for electroporation.

In a preferred embodiment an apparatus comprises particles adapted according to the invention; means to add particles to a liquid sample, and means to apply an electric field to the sample. The apparatus may comprise a flow system having a flow cell within which the liquid sample may be exposed to an electric field.

According to a further aspect the invention provides a method for enhancing an applied electric field in a region of a cell, comprising: providing at least one particle to the cell, the particle having a high permittivity, being conductive or having a conductive core and being adapted to associate with the cell; and applying an electric field to the cell.

Preferably the said at least one particle is adapted as described herein. Preferably at least two particles are provided to a cell, the particles being adapted to act together to enhance the electric field in their vicinity. In preferred embodiments a first particle adapted to enter the cell is provided, and a second particle adapted to bind to a target molecule on the exterior of the cell membrane is then provided, so as to provide an arrangement of a first particle on the inside of the cell membrane and a second particle external to the cell membrane, in proximity to the first, so causing enhancement of the applied field across a portion of the cell membrane.

It is further understood that features described for given aspects of the invention or embodiments are not intended to be employed in that aspect or embodiment only, rather they may be combined to achieve the purposes of the invention.

In some embodiments particles may be adapted to remain associated with target cells for an extended period after administration. Thus in accordance with any of the aspects of the invention herein, the method may comprise administration of particles followed by multiple instances of application of the electric field at intervals after administration of the particles.

The present invention will now be described, by way of examples only, and with reference to the Figures in which:

### BRIEF DESCRIPTION OF THE FIGURES

FIG. 1 shows a plot illustrating the effect of the said method on bacterial spores. The effect is monitored as the release of DNA due to electrolysis;

FIG. 2 shows a plot of colony forming unit (CFU) of two populations of bacterial spores where one has been exposed to the method and the other has not (control);

FIG. 3 shows the time course of pores formed in a cellular membrane;

FIG. 4 shows the field flux concentration from a microsphere in a non-homogeneous electric field;

FIG. 5a shows enhancement of the applied field by an arrangement of two particles, showing particular enhancement in the region between the two particles.

FIG. 5b shows enhancement of the applied field by a single particle.

FIG. 6a is a diagrammatical view of a single particle, having a high permittivity or comprising a conducting core, and how this concentrates an electric field adjacent to cell membrane;

FIG. 6b is a diagrammatical view of a particle bound to target molecule at the target cell surface by a ligand forming part of a coating;

FIG. 6c is a diagrammatical view of a particle spaced from the ligand by a linker;

FIG. 6d is a diagrammatical view of a pore opening as a result of field enhancement adjacent to a particle associated with a target cell membrane;

FIG. 7a is a diagrammatical view showing field enhancement by a single particle located inside the cytoplasm of a cell.

FIG. 7b is a diagrammatical view of a single particle located at the nuclear membrane, concentrating the field in a region adjacent the nuclear membrane;

FIG. 7c is a diagrammatical view of a configuration adapted for nuclear electroporation comprising a particle associated with the nuclear membrane and second particle associated with the cell membrane;

FIG. 8a is a diagrammatical view showing how a first particle inside and a second particle outside the cell membrane give additional field enhancement across the membrane;

FIG. 8b is a diagrammatical view showing how a first particle inside and a second particle outside the cell membrane give additional field enhancement and the resulting opening of a pore;

FIG. 9a shows two particles having a high permittivity or being conductive, (such as metal particles), may bind to the exterior of a cell and jointly produce an enhanced field in the vicinity of the cell membrane;

FIG. 9c shows how two particles bound to the exterior of the nuclear membrane, that are closely located, produce field enhancement in a region of the nucleus sufficient to lead to electroporation of the nuclear membrane;

FIG. 10a shows diagrammatically the significance of field orientation relative to orientation of pairs of particles and how a first field direction E1, for a first pair of particles, produces a greater effect of field enhancement on the cell membrane than a second pair of particles, for a second field orientation E2, produces a greater effect;

FIG. 10b is a diagrammatical representation of random provision of particles to a number of target cells in a region of tissue and shows for a first field direction E1 certain particle pairs or arrangements produce a greater effect than others;

FIG. 11a shows in diagrammatical form treatment of target cells, within a region of tissue within the body, for example a tumour, using planar electrodes external to the body;

FIG. 11b shows in diagrammatical form treatment of target cells, with the use of electrodes, implanted in the body, and illustrates the effect of different field orientations on different orientation of particle arrangements;

FIG. 11c shows in diagrammatical form treatment of target cells at a region within the body;

FIG. 11d is a diagrammatical cross section, through a body during treatment, of target cells in a region of the body;

FIG. 11e is a diagrammatical cross section through a body during treatment of target cells in a region of the body, showing a movable electrode, for example a hand-held probe, which may be separated from the skin by an insulating layer 116;

FIG. 12a is a diagrammatical overview, (showing a horizontal cross section through the body of a patient), of an apparatus for moving electrodes around the patient's body during treatment;

FIG. 12b shows a means for displacing electrodes along the length of a patient's body as well as around the body so as to perform treatment in accordance with the method;

FIG. 12c is a diagrammatical overview of an apparatus and shows multiple electrodes around a region of the body, adaptable to the contours of the body, for example by way of a flexible support structure that is adapted to deform to conform to a patient's body;

FIG. 12d is a diagrammatical overview of a portion of the apparatus in FIG. 12c, that supports the multiple electrodes, removed from the body;

FIG. 13a is a flow diagram showing steps in the method, with a first particle type adapted to enter the target cells and a second particle type adapted to bind to the surface of the target cells;

FIG. 13b is a flow diagram depicting an example of the method where a single particle type is used, the particle being adapted to enter the target cell and reside at the surface of the cell before endocytosis;

FIG. 14 shows a diagrammatical view of a system for treating target cells in blood; particles are administered into the body and allowed to associate with the target cells and blood is passed through a flow cell where they are exposed to an electric field before returning blood to the body;

FIG. 15a shows an alternative embodiment, with a cell culture of attached cells and particles added to the culture, and in which a field is applied across the cell layer, so that target cells are affected and non-target cells are unaffected;

FIG. 15b is a diagrammatical view of a cell culture of cells in suspension in a bioreactor, and shows particles mixed with cells in a liquid medium, flowing through a flow cell in which cells are exposed to a field before being returned to the bioreactor;

FIG. 16 is a diagrammatical view of an alternative embodiment of the invention adapted for sample processing, for example for use in diagnostics;

FIGS. 17a to 17d show steps in a method for introducing a species into a target cell by means of reversible electroporation according to the invention, and

FIGS. 18a to 18d show steps in a further method for introducing a species into a target cell by means of reversible electroporation according to the invention.

### DETAILED DESCRIPTION OF PREFERRED EMBODIMENTS OF THE INVENTION

In the following description the terms particle, microparticle, microsized particle and nanoparticle are used interchangeably.

Appropriate dimensions and morphology of particles are described by way of example only.

### Example 1

### The Effects of Varying Bead Concentration (2, 0.5 and 0 μl) on Spore Electrolysis Efficiency

One hundred mg of Biobit Bacillus thuringiensis subsp. kurstaki containing 3.2×109 spores/g (Valent BioSciences Corp, Libertyville, USA) was re-suspended in 1 ml of demineralized water and centrifuged for 90 sec. at 12000 rpm. This procedure was repeated 4 times. The supernatant was discarded. The final solution contains approximately 3.2×108 spores. This solution was diluted to a final concentration of 3.2×105 spores/ml. and subsequently 12 μl spore sample was used for electrolysis and PCR.

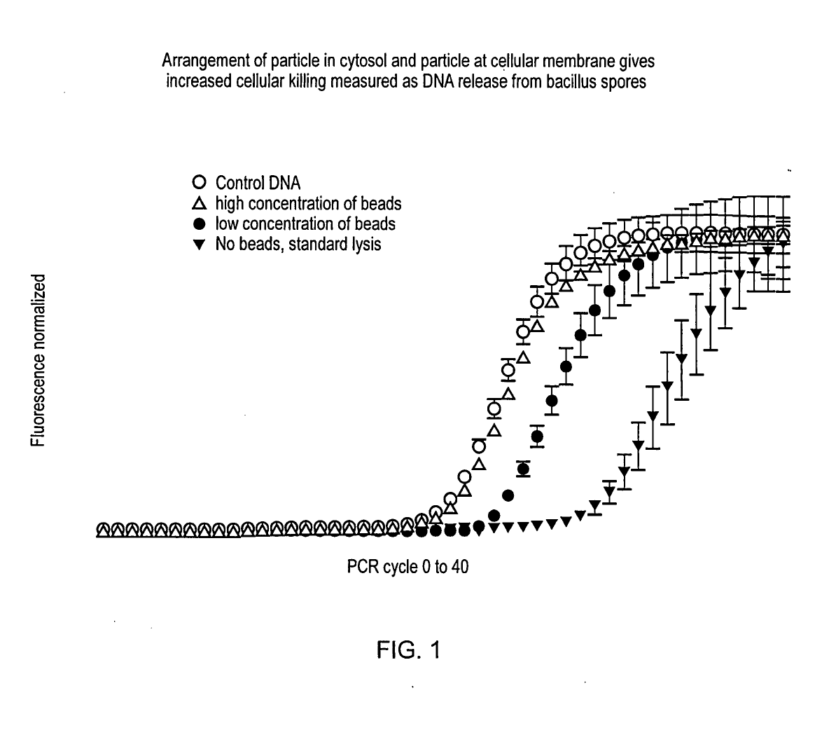

Voltage, time and frequency were kept constant (at 10 V, 30 sec and 100 KHz, respectively) variations was made in the concentration of iron oxide beads that was added respectively 2 and 0.5 μl 1 μM iron oxide silica coated beads (Merck). FIG. 1 shows the results of this experiment and as apparent, the high concentration of bead of 2 μl to the 12 μl spore sample showed a decrease in CT (threshold cycle) compared to standard lysis without beads, thus demonstrating release of amplifiable DNA from the spores. Lowering the bead concentration to 0.5 μl decreased the effect considerably.

It other experiments it was shown that addition of more than 0.5 μl silica coated iron oxide beads directly to a 20 μl PCR reaction gave more than 50% in the PCR yield. The above experiments was carried out with a final bead addition of respectively 1/12×2=0.17 μl bead to a 20 μl PCR reaction for the high concentration of beads and a 1/12×0.5=0.04 μl bead to a 20 μl PCR reaction for the low concentration of beads. Therefore, it should be expected that less PCR inhibition is experienced in the low concentration of bead than in the high concentration. The results are opposite that, thus the effect of the increased beads is exceeding the PCR inhibition.

It is believed that the effect of the increased bead concentration is to increase the likelihood of forming two particle arrangements on opposing sides of the spore leading to an increased field flux between the particles with concomitant molecular disarrangements in the coating of the spores and cell wall leading to killing of the spore.

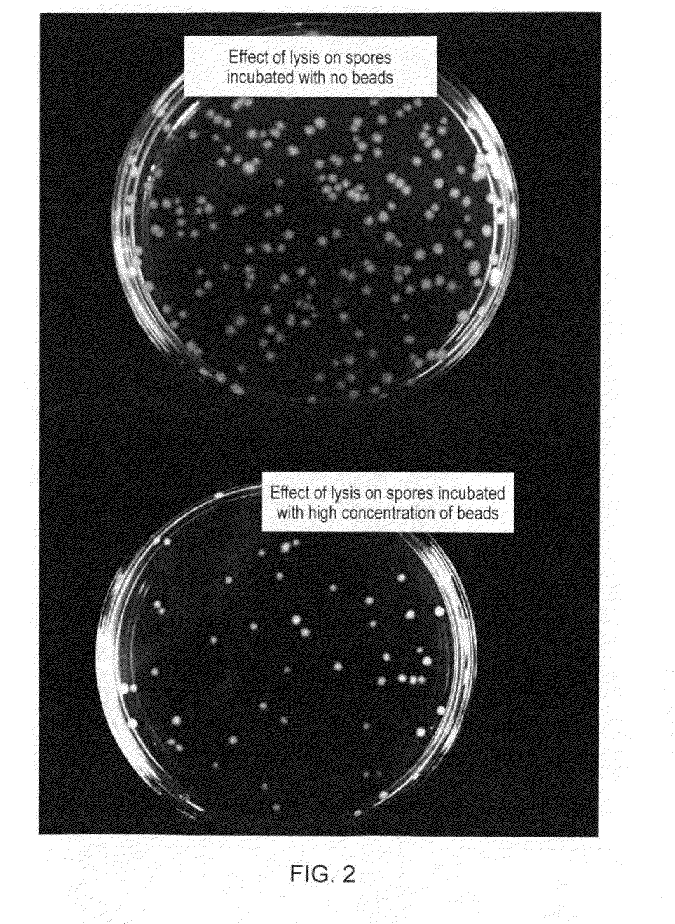

FIG. 2 shows the spores grown on agar plates subsequent to the lysis and spores incubated with beads and grown show no effect whereas the number of colony forming units from the sample undergone the electrolysis in presence of beads shows a marked reduction (95% less CFU's compared to control).

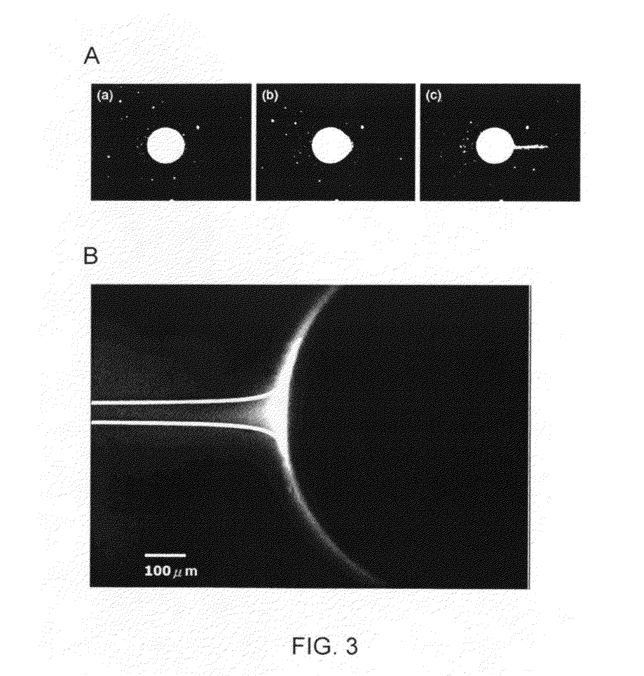

FIG. 3 illustrates the field flux from a sphere formed particle in a non-homogenous electrical field. The field flux increase causes fluorescent ions to move in the most concentrated region of the flux and electromotion overcomes diffusion and a concentrated stream of fluorescent ions is radiating out from the sphere. The same principles are governing smaller particles and can be used to generate a electric flux concentration between two particles with associated molecular rearrangement for any charged molecule within the concentrated flux region.

A) The sequential images of microparticle concentration evolution for a cation exchange granule with a step change in the field to 100 V/cm, using co-ion fluorescent-dye tagged microspheres in 10 mM Tris buffer (pH 8) at very low density (5.0×10E6 particles/mL). The images are taken at 0 (a), 1.35 (b), and 2.44 s (c).

B) Ion-concentrated ejection cone. The yellow profile is the theoretical prediction of:

R/a=(√3/┌0.75)(1−(a−r)3)−0.5,

based on flux balance within the two bounding pole field lines.
R: radius of jet ejecting from the sphere
r: radius of sphere
a: area of sphere
┌: field in volt per cm

This principle is also governing smaller particles and can be used to create a electric flux concentration between two particles with subsequent molecular disarrangement of charged molecules between the two particles. In the case of a cell membrane intercalated between the two particles it will lead to pore forming events in the cellular membrane. (Biomicrofluidics 2008, 2, 014102)

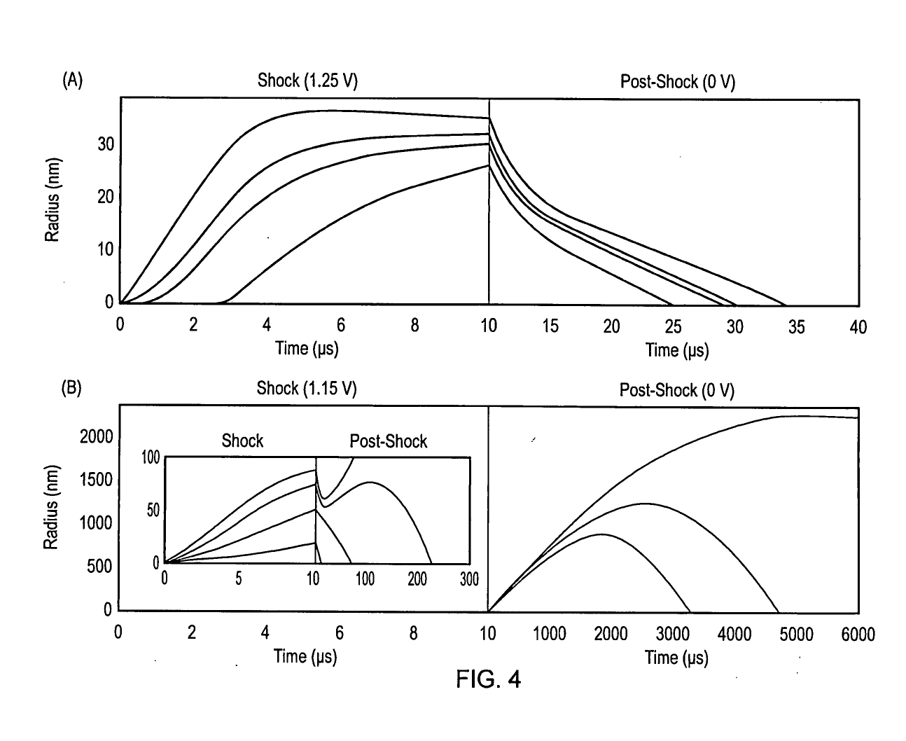

FIG. 4 illustrates one important outcome of pore forming events in a cellular membrane caused by a single electrical pulse. In general high amplitude electrical pulses can cause formation of many small pores that reverses over time and the membrane re-seals. However, a smaller pulse creates a smaller amount of pores of and the newly formed membrane pores can undergo a process called “coarsening” where the resealing eventually does not occur and the pores forms a single very large pore that eventually leads to cell death. This information is important because it illustrates that lower voltage can be used to cause more damage than high voltage, so the optimal effect is not necessarily achieved by the highest amplitude and highest frequency.

Exposing a cell to a lower voltage gives a relative higher number of irreversible pore forming events compared to exposure with a higher field. The higher field generates many pores but with smaller diameter and they are reversible. The figure illustrates pore radii during a 10 μs pulse and the post shock evolution of pores. The gray scale represents the pore radii distribution (i.e., the number of pores with radii between r and r+dr). Solid lines show the 10, 20, . . . , 100th percentiles of the maximum pore radius, illustrating the evolution of the pore radii in time. (A) Evolution of pores after a 1.25 V pulse, which created 18,025 pores. After the pulse, all pores shrink to rm (the minimum-energy radius of (B) Evolution of pores after a 1.15 V pulse, which created a smaller number of pores, 2772. After the pulse, all pores shrink to rm except the largest pore, which grows to a stable radius of 2.23 μm. (Inset) The pulse and the first 300 μs after the pulse shown on an expanded vertical scale. (Redrawn from Biophys J. 2004 May; 86(5): 2813-2826).

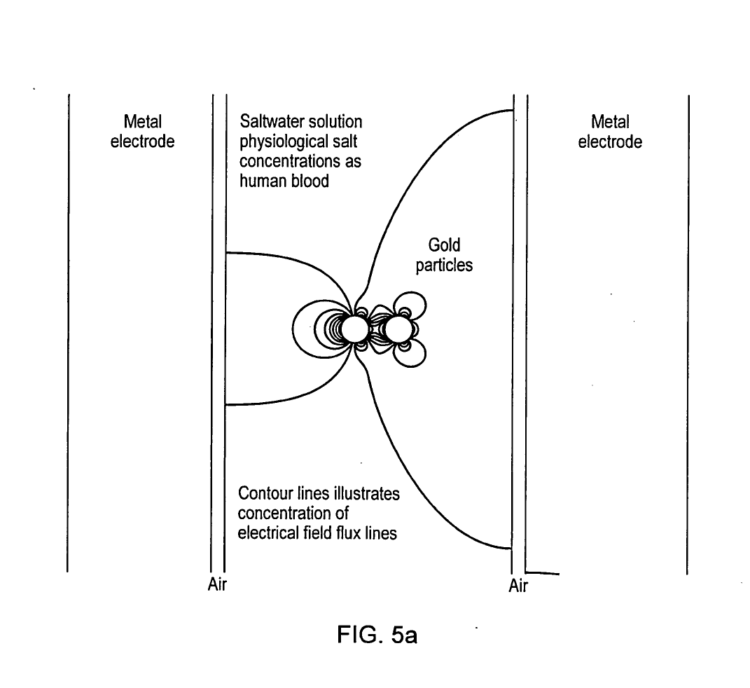

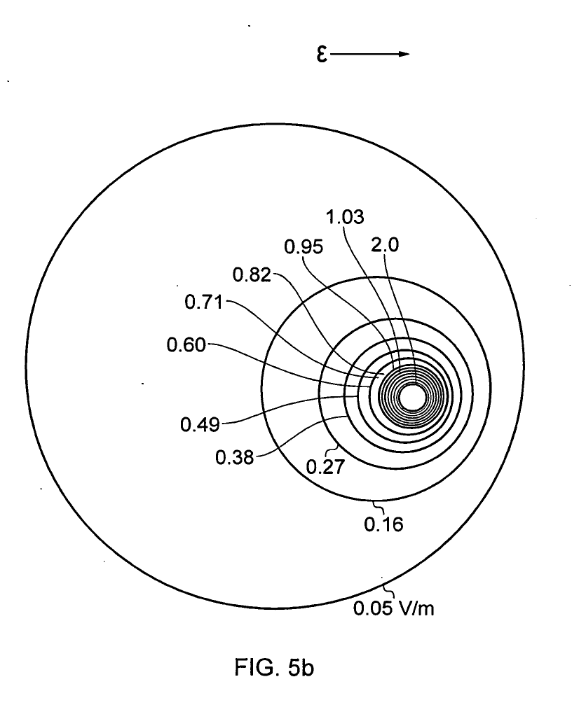

FIG. 5a illustrates the field enhancement effect of a pair of adjacent conductive particles. FIG. 5a shows a plot of results from a theoretical model of electrical field flux lines around two gold particles in salt water that has chemical resemblance to human blood. The setup has two electrodes with a voltage difference of 300 V separated from the solution with a gap of air. The particles are in the solution. The plot illustrates the field flux concentration effect that can be achieved by positioning two gold particles in proximity of each other. The field is highly concentrated in the region between the two particles. Other metals give similar results. In the model the particles were 1 um diameter gold microspheres. Substantially similar results were achieved for 60 nm and 40 nm diameter gold microspheres.

FIG. 5a illustrates the optimal arrangement between two particles where one is located in the cytoplasm of a mammalian cell and one on the extracellular side of the membrane. The cell and cell membrane is not shown since the electrical permittivities are not of significant magnitude to influence the field line concentrations achieved by the two particles in this simulation. A cell membrane located between the particles would be exposed to a high field region, leading to pore formation. A similar arrangement is optimal for destruction of a spore but where the particles are found on opposing sides of the spore with the same result as the effect described for the mammalian cell.

FIG. 5b illustrates field enhancement by a single particle, in this case modelled as a silver particle of diameter 500 um immersed in physiological saline, of relative permittivity 88, in a low uniform field of 0.1V/m. Contours of field strength around the bead are shown in V/m. It can be seen from the figure that a field enhancement of over 20 times is achieved adjacent to the surface of the bead, and approximately 10 times at a distance from the bead surface approximately equal to the radius of the bead. Simulation used the software COMSOL3.5. The degree of field enhancement for smaller beads such as micro- and nano-beads is expected to be at least equal to these factors.

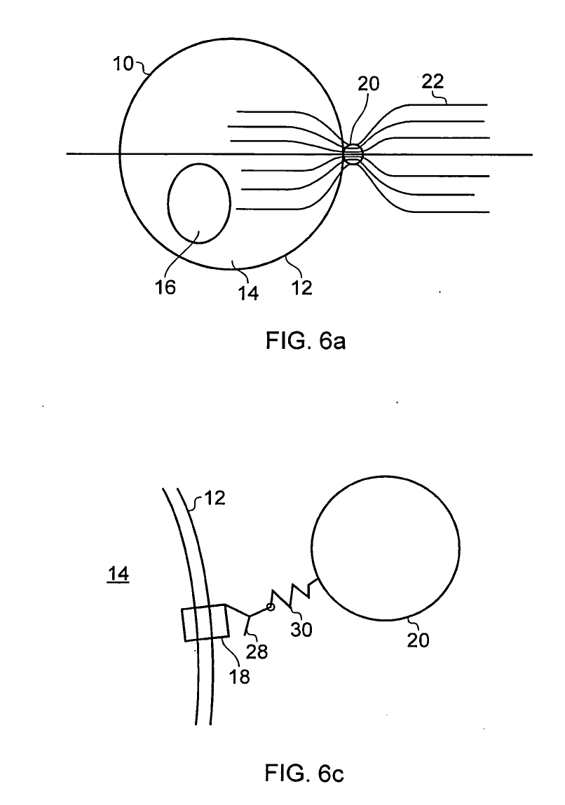

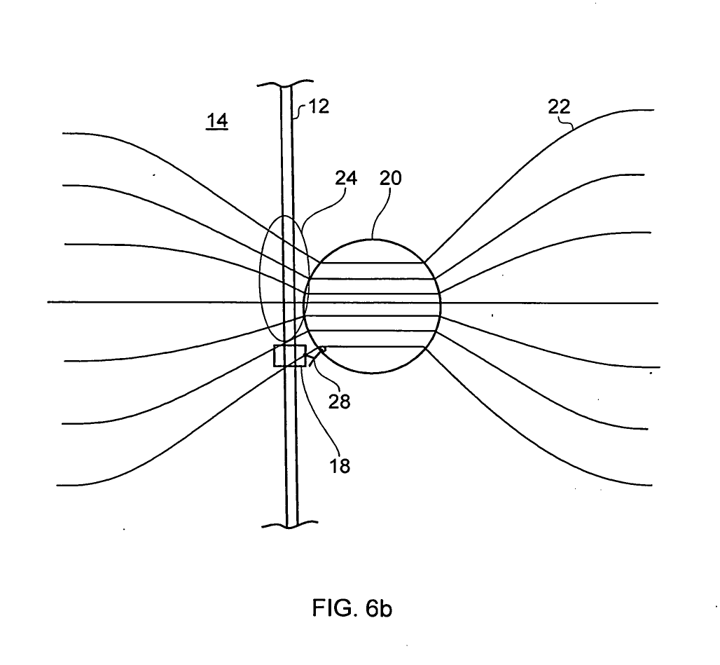

FIG. 6a shows an arrangement resulting from an embodiment of the method of the invention in which a cell 10 having a membrane 12, cytosol 14 and nucleus 16 has a particle 20 that is either conductive or has a high permittivity associated with the extracellular side of the cell membrane. When an electric field, in a direction indicated by the symbol E and shown by means of field lines 22, is applied to the cell and the surrounding medium the field is enhanced by the particle in a region 24 incident on the cell membrane.

Enhancement of the field is shown by means of the increased concentration of field lines in the vicinity of the particles compared with in the environment surrounding them (note the appearance of field lines in the extracellular medium is symbolic and not intended to be an accurate representation, in particular the effects of the charge double layers and impedances at membranes are omitted). With appropriate choice of field strength and/or time variation the enhanced field in the region 24 is sufficient to cause reversible electroporation of the membrane in this region, but is below the threshold for irreversible electroporation. If a higher field strength and a different time course of the field are selected the enhanced field exposure is above the threshold for irreversible electroporation, leading to death of the cell.

FIG. 6b shows a preferred version of the embodiment in which the particle 20 is bound to a target molecule 18 on the cell surface by means of a ligand 28 provided as part of a coating on the particle. The ligand 28 may be chosen to be specific for target molecules 18 that are present only or preferentially on target cells, so allowing targeted binding of particles 20 to target cells and no, or lesser, binding to non-target cells. The ligand may in some embodiments be adapted to bind to a specific region of a target molecule, for example an extracellular region of a transmembrane protein.

More than one ligand type may be present on a particle, and the ligands may be targeted to the same or different target molecules or target molecular regions. The target molecule may be any molecule, such as a protein, protein complex, or sugar. The target molecular region may for example be a region of the protein or one protein in a complex. The embodiment in FIG. 6b therefore provides novel targeted electroporation or destruction of target cells in a mixed cell population mediated by dielectric, high permittivity, or conductive particles.

In a further preferred version of the embodiment shown in FIG. 6c a ligand 28 may be attached to the surface of the particle by a linker molecule 30, which gives the advantage of controlling the mean distance of the particle from the lipid bilayer of the cell membrane, so controlling and optimising the effect of field enhancement on pore formation in the membrane.

Particles in this embodiment are preferably of higher permittivity and/or conductivity than the mean permittivity and/or conductivity of the environment in the region surrounding them, that region comprising one or more of the extracellular fluid; extracellular matrix; cell membranes of other cells; cell surface molecule such as membrane proteins and sugars. The conductivity of the surrounding medium is therefore a composite conductivity derived from the presence and conductivities of the various components, and therefore may take a range of values up to that of physiological saline or blood. The effective permittivity of the surrounding medium is a composite of the various component permittivities in a similar manner.

Particles may have characteristics as disclosed herein, for example being dielectric particles, conductive particles or particles having a dielectric or a conductive core, for example a metal core, such as gold, and a coating comprising at least one ligand targeted for a target molecule on the cell membrane. Such ligands may comprise antibodies, aptamers, protein binding partners and peptides, as known in the art.

FIG. 6d shows a diagrammatic representation of a pore forming event in the cell membrane 12. An applied field shown by the field lines 22 is concentrated by particle 20, bound to a target molecule 18 in the cell membrane by means of a ligand 28, shown here in the form of an antibody but which may comprise other species adapted to bind to a target molecule. The locally enhanced field opens at least one pore 25 in the membrane through which species 26 to be introduced into the cell may move, by diffusion and/or electrophoresis as known in the art. Without wishing to be bound by any one interpretation, it is expected that for a high affinity binding of the ligand 28 to the target molecule 18 that the particle 20 will in general remain associated with the outside of the cell membrane while other, unbound species 26 may enter the cell. The parameters (such as strength, pulse shape and duration, polarity) of the field are chosen such that pore 25 closes when the field is removed. In this way reversible electroporation is achieved at a field applied to electrodes remote from the cell much lower than the threshold field for electroporation without particles present and associated with the cell membrane.

Species 26 may comprise for example a drug, a protein, a nucleic acid such as DNA or RNA, for example a genetic construct as used in transfection of cells, for example in gene therapy, or a RNA construct used in for example gene silencing. Species 26 may be the substance itself in ‘naked’ form, the substance encapsulated in a shell or coating, or bound to or contained within a further entity such as a particle. The species may be charged or uncharged and in a preferred embodiment is adapted for electrophoretic motion within an applied field, as known in the art of electroporation. The species may be adapted to be targeted to a specific target location or target molecule within the target cell, for example located on the nuclear or mitochondrial membrane. The species may be adapted to enter the nuclear membrane, or may be adapted to associate with a target molecule, for example a nuclear receptor, on the surface of the nuclear membrane. The species may comprise a particle adapted to enhance electroporation as described herein, when in situ in the vicinity of a membrane, for example the nuclear or a mitochondrial membrane.

In a further preferred embodiment, the coating species 28 and the target molecule 18 may be selected to have a moderate or low binding affinity, such that they may dissociate when a field is applied. In this way, particle 20 is able to dissociate from its binding position at the exterior of the cell membrane and enter the cell via pore 25. In this embodiment particle 20 may comprise the species to be introduced into the cell bound to its surface and may act as a carrier to introduce the species into the cell.

In a further embodiment as described previously, the applied field strength and time profile are selected to cause the pore 25 to form irreversibly, leading to cell death.

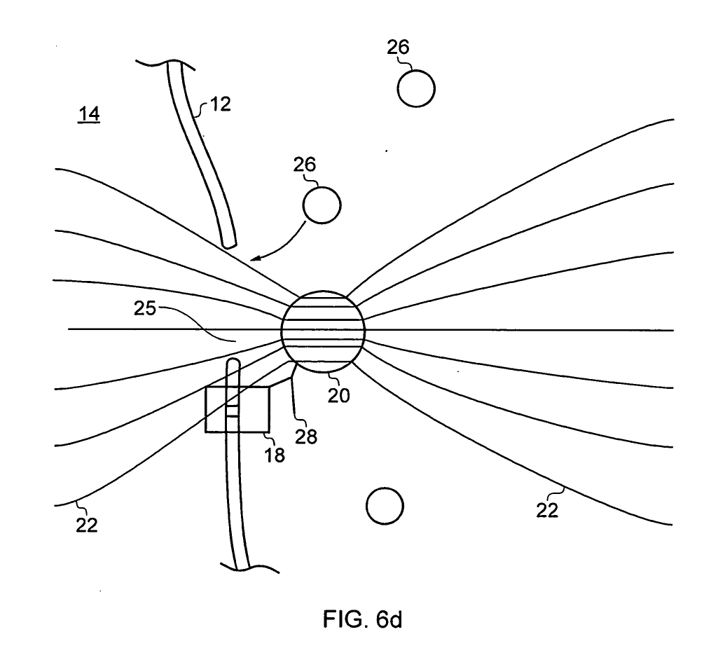

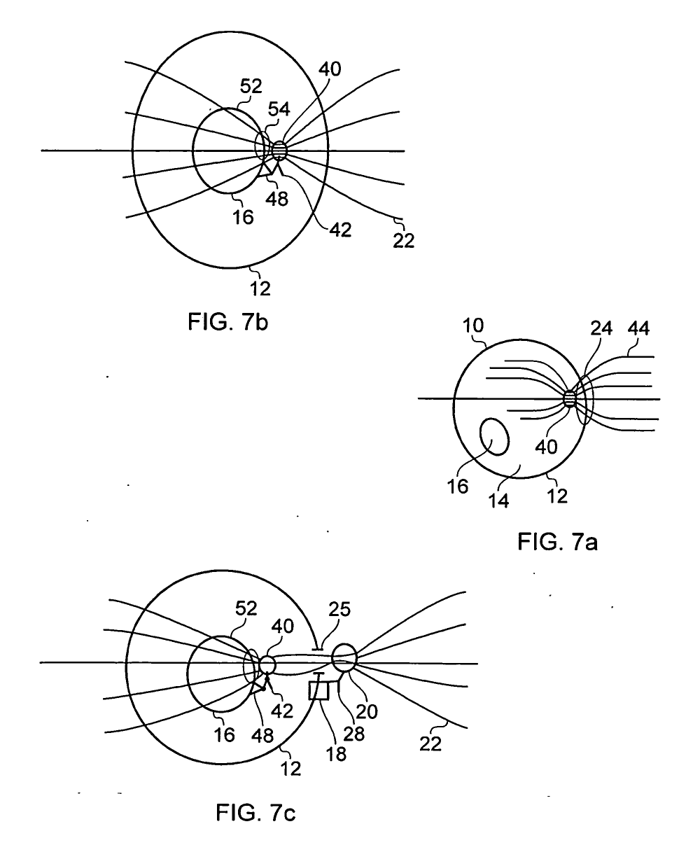

FIG. 7a shows an arrangement resulting from use of a further embodiment of the method of the invention, which comprises providing particles adapted to enter a target cell, allowing at least one particle to enter the cytoplasm, and then exposing the cell and at least one particle to an electric field, so causing enhancement of the field in the vicinity of the particle resulting in electroporation of the cell membrane. A cell 10 as described before now has a particle 40 provided within it, which may be a dielectric particle, in some embodiments of high permittivity as described above, in alternative embodiments may be conductive, for example comprising a metal, for example gold. As is shown in FIG. 7a a particle within the cell close to the membrane produces a region 24 of enhanced field in the membrane in the vicinity of the particle, so reducing the threshold for electroporation. The effect is greater for particles close to the membrane than for particles further away in the cytosol. In preferred embodiments the particles 40 comprise a coating adapted to promote association with target molecules on the interior of the cell membrane 12 or are present in sufficient quantity within the cytosol that at least one particle will be located close to the membrane without being specifically targeted to it. Particles may comprise a coating that facilitates entry to the cell, for example by means of endocytosis, and the coating may comprise one or more ligands that confer specificity of the endocytosis process to target cells as described further herein.

In one embodiment the applied field and its time profile are selected to cause irreversible electroporation of the cell membrane and hence cell death. In further embodiments the particle is adapted to associate with a target molecule within the cell, for example located on an internal membrane such as the nuclear membrane or a mitochondrial membrane, the particle acting to enhance an applied field resulting in death of the cell from non-thermal means, for example apoptosis or necrosis following damage to an internal organalle or structure of the cell.

In further embodiment as shown in FIG. 7b the method of the invention is applied to cause electroporation of the nuclear membrane resulting from enhancement of an applied electric field by particles bound to the nuclear membrane. FIG. 7b shows a local field enhancement in region 54 of the nuclear membrane caused by particle 40 bound to it, the particle 40 preferably comprising a coating having a ligand 42 adapted to associate with a target molecule 48 on the nuclear membrane 52. The method in this embodiment comprises steps to introduce particles 40 into the cytoplasm, for example by endocytosis, conventional electroporation, or particle-mediated electroporation of the cell membrane as described herein, followed by an incubation time for particles 40 to reach the nuclear membrane and associate with target molecules 48.

In an embodiment directed to reversible electroporation a species to be introduced into the nucleus may be introduced into the cytoplasm along with the particles 40 or in a subsequent step. In one embodiment the particles 40 are adapted to enter the nucleus as part of the electroporation process, and may comprise or carry a payload to be introduced into the nucleus, for example as a surface coating or interior content. The field needed to cause electroporation of the nuclear membrane in the absence of particles is high as the impedance of the cell membrane appears in series with that of the nuclear membrane, which leads to low efficiency and damage to cells. With the use of particles as mediators to reduce the threshold for reversible electroporation, the required field is lower and viability of cells following nuclear electroporation is higher. In embodiments directed to causing cell death, field parameters are chosen such that electroporation of the nuclear membrane leads to apoptosis or necrosis of the cell. In a further embodiment, species may be targeted to and delivered into mitochondria using a similar method.

In a further embodiment shown in FIG. 7c, particles 20 are provided additionally targeted to the cell membrane as described above, and act to cause pore-forming events in the cell membrane when the electroporation field is applied. One or more reversible pores 25 effectively short-circuit the resistance of the cell membrane 12 so greatly reducing the applied field needed to achieve nuclear electroporation. Species to be introduced to the nucleus are provided within the cell cytoplasm and diffuse or move electrophoretically, as for example in the case of a DNA construct, through the field-opened pore in the nuclear membrane and into the nucleus.

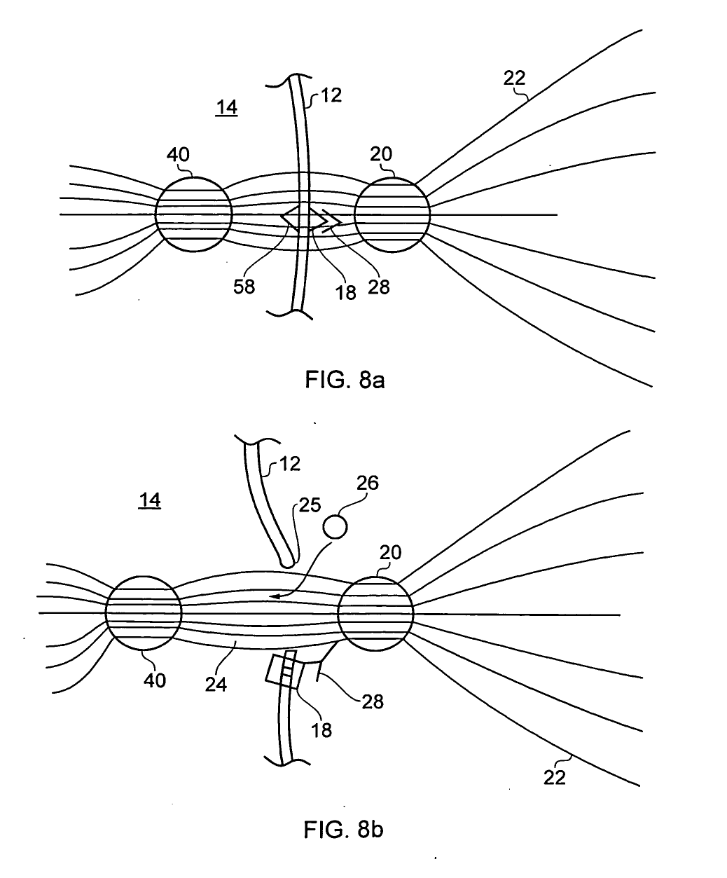

FIGS. 8a and 8b show a further arrangement of particles and target cell arising from a further embodiment of the method according to the invention. As shown in FIG. 5a, it has been found that two particles may act co-operatively to create a significantly greater enhancement of an applied field than a single particle alone.

FIG. 8a shows a portion of the membrane 12 of a cell having a first particle 40 within the cytosol and a second particle 20 bound to a target molecule 18 on the exterior of the cell membrane 12 by means of a ligand 28 as described before. The two particles may be dielectric particles and may have a high permittivity, may be conducting or have a conductive core, for example comprising a metal such as gold or a metal oxide, such as Fe₃O₄. The two particles cause an enhancement of the electric field in their vicinity, and especially between them, and in any region of the cell membrane adjacent to them, shown here as a region 24 between the particles.

In preferred embodiments of the invention, first particles 40 are adapted to enter the cell, and second particles 20 are adapted to bind to the exterior of the cell. One or both of the first and the second particles may be targeted to target cells as described herein. The first particles 40 may be adapted to enter target cells preferentially, and may be adapted to bind to a specific location or range of locations with the target cell, for example to a target molecule located on or adjacent to the inside of the cell membrane. Alternatively, the first particles may be simply adapted to enter both target and non-target cells. The second particles are preferably adapted to bind to target molecules on the exterior of the target cells as described herein.

The situation resulting from the method of the invention in this embodiment is that target cells have a particle arrangement associated with them, comprising at least one first particle within the cell and at least one second particle bound to the exterior of the cell membrane, as shown in FIGS. 8a and 8b. The adaptations of one or both of the particles to associate with or to enter target cells selectively are such that non-target cells do not have this particle arrangement.

In a preferred embodiment the first, interior particle 40 is adapted to bind to an interior region 58 of a transmembrane protein for example a receptor or ion channel, so as to be located close to the second, exterior particle 20, and the first particle has a coating comprising ligands adapted to associate with region 58. In one such embodiment the interior particle 40 is adapted to bind to the intracellular region of a transmembrane protein and the second, exterior particle 20 is adapted to bind to the extracellular region of the same transmembrane protein.

FIG. 8b shows diagrammatically a pore 25 opening in the membrane 12 under the influence of the high field region 24, and a species 26 to be introduced into the cell entering through the pore.

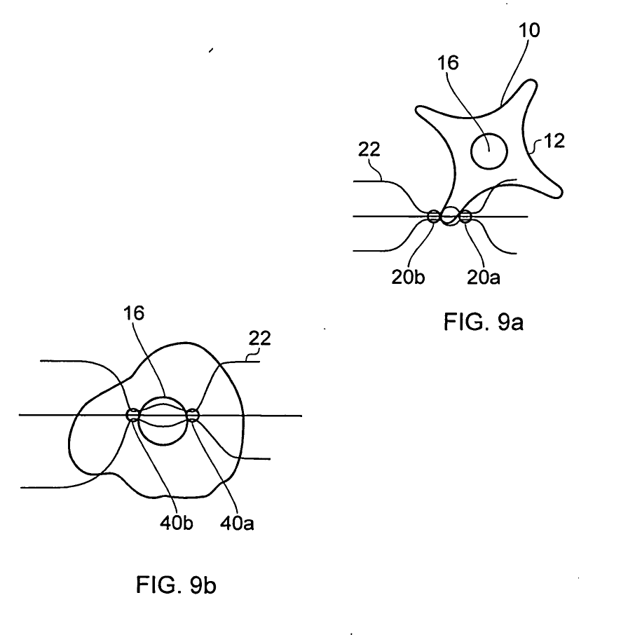

FIG. 9a shows an arrangement of particles arising from a further embodiment of the invention, in which at least two particles 20a, 20b are bound to the exterior of a target cell. Enhancement of the applied field occurs when the particles are located in proximity to one another. Regions of enhanced field intersect the cell membrane according to the position of the two particles, the shape of the cell, and the orientation of the particles with respect to the applied field. Such a particle arrangement may arise in particular at a region of a target cell with higher aspect ratio, for example a process or outgrowth, for example as in neuronal cells. While only two particles are shown it will be appreciated that in this embodiment, the method advantageously provides sufficient particles to the target cell that there will be on average at least one such arrangement of particles associated with the cell.

FIG. 9b shows an arrangement of particles arising from a further embodiment of the invention, in which the particles are adapted to enter the cell and to associate with the nuclear membrane, so causing field enhancement across a region of the nucleus and nuclear membrane, resulting in electroporation of the nuclear membrane. Depending on the applied field parameters, electroporation may lead to disruption of the nuclear membrane or nuclear function, leading to cell death, or may be reversible.

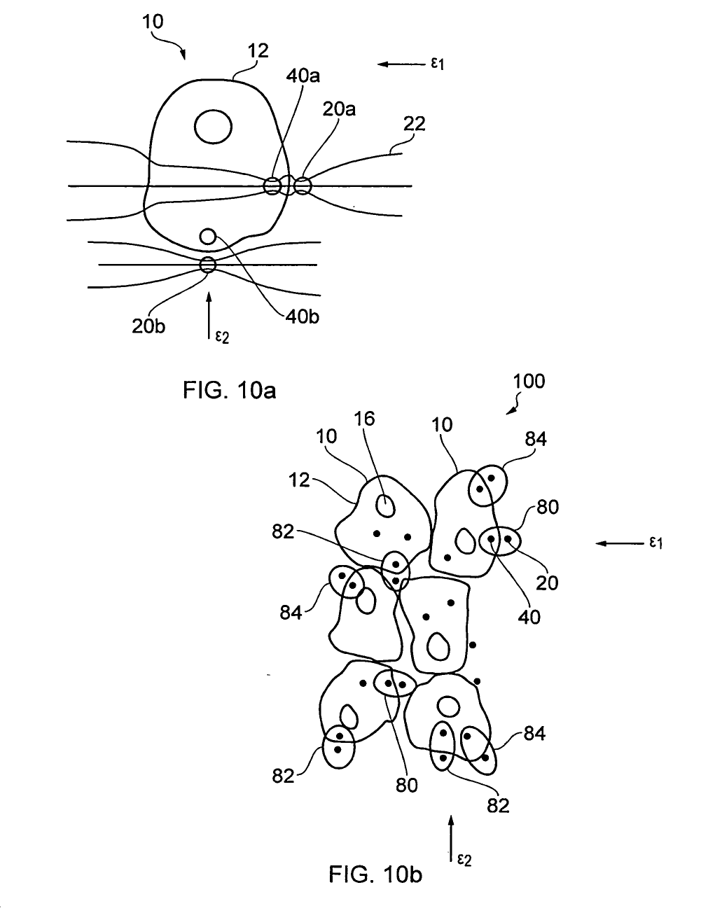

In a further embodiment of the invention, the orientation of the electric field may be controlled or varied with respect to the target cells, for example a group of target cells such as a tumour, or the body of a subject hosting the target cells. FIG. 10a shows an arrangement of particles arising from an embodiment of the invention, and illustrates that the orientation of the field with respect to one or more target cells may in some embodiments affect the degree of field enhancement. In FIG. 10a a first particle arrangement 20a, 40a has the axis of the particle arrangement—the line joining the two centres of the particles—aligned with the field direction E1. This is expected to lead to a higher degree of field enhancement in the region of the membrane near or between the particles. A second particle arrangement 20b, 40b has its axis perpendicular to the E1, which is expected to lead to lesser field enhancement in the region of the membrane near or between the particles. Field in direction E2 reverses this situation. It is clear that for a random orientation of particles arrangements around a target cell, the best chance of achieving high field enhancement is through using multiple orientations of the applied field.

FIG. 10b shows an arrangement of particles arising from a further embodiment of the invention, the particles being provided within a region of tissue 100 comprising a number of target cells 10. Particles targeted to these cells will in general be distributed randomly and so within the tissue particle arrangements 80, 82, 84 resulting from the invention will be oriented randomly. For a first field direction E1 certain particle pairs or arrangements (80) will produce a greater effect than others (82). For a second field direction E2 the reverse is true. Some particle arrangements (84) will have an intermediate level of effect for both field directions. Therefore varying the orientation of the applied field with respect to a group of target cells, or the tissue or body of a subject in the case of treatment of disease, is advantageous and may be achieved by the method and apparatus of the invention.

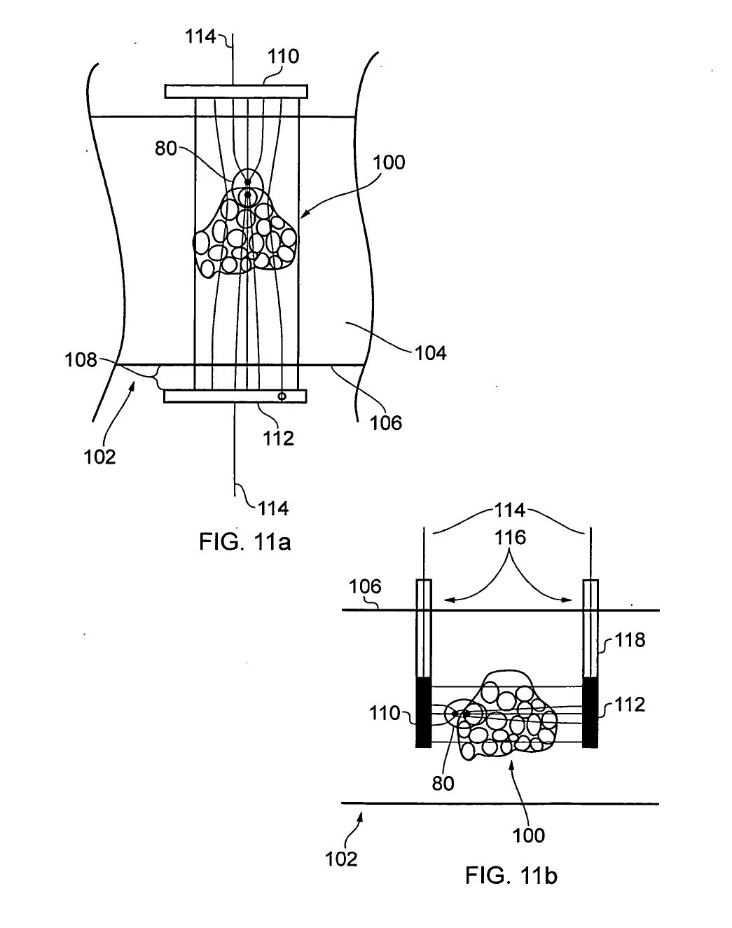

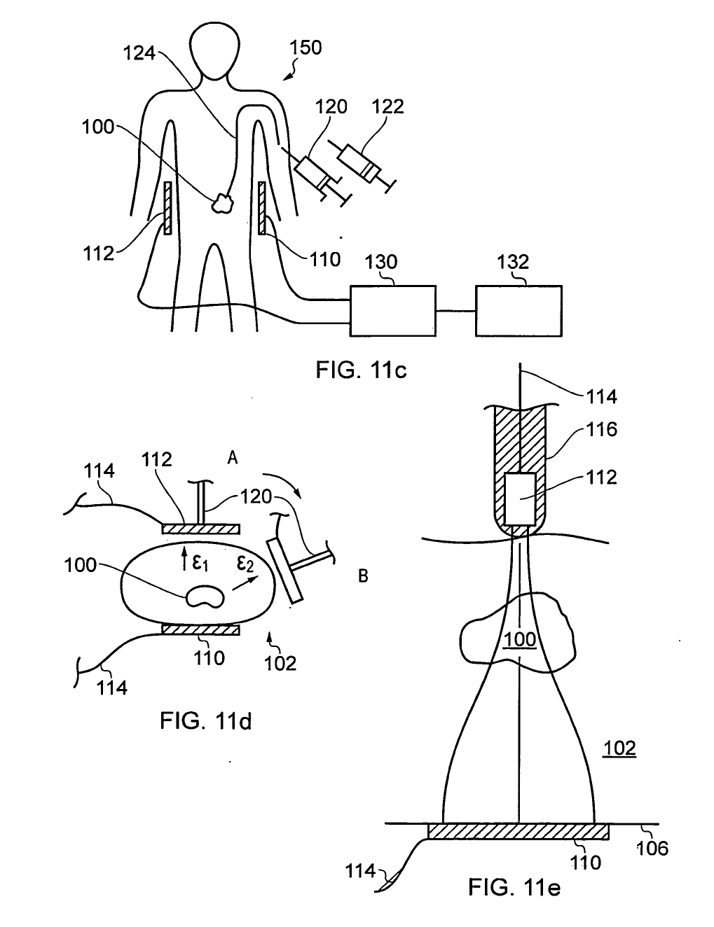

In accordance with the invention, the electric field may be applied by electrodes disposed in a variety of ways around the body. In contrast with prior art methods, the field enhancement of the invention allows a greater variety of electrode placement to be used. Electrodes may be located external to a region comprising the target cells, such as a container, tissue, or body of a subject. It is a particular advantage of the invention that the applied field can be lower than in the prior art, and some embodiments use electrodes placed externally to the body of a subject. For example, FIG. 11a shows treatment of target cells within a region of tissue 100, for example a tumour within the body 102 of a subject, the field being applied by a first electrode 110 and a second electrode 112, connected to a source of potential, such as a device according to the invention by means of connections 114. In this embodiment the electrodes are planar electrodes external to the body. The electrodes are shown separated from the body by a gap 108. In an alternative embodiment one or both electrodes may be in contact with the skin 106. The electrodes may be flexible or adapted to conform to the contours of the body, or may be shaped to maintain a given separation from the body.

FIG. 11b shows treatment of target cells within a region of tissue 100 using implanted electrodes 110 and 112, each forming part of implanted probes 116. The probe 116 has one or more conductive regions adapted to provide a region of electric field within the body and one or more insulated regions 118. Probes suitable for use in this mode are known in the prior art.

FIG. 11c shows a method and an apparatus for treatment of a disease within a subject by means of electroporation of target cells at a region within the body. Particles are administered systemically to a subject 150 for example by means of injection or infusion into the blood stream. According to the method of the invention, a composition 120 comprising a first particle type is administered, the particles travel to the region 100 through the circulatory system 124, and associate with the target cells. After a chosen time interval t1, chosen to allow particles to reach their desired locations and arrangement(s) with respect to the target cells, the species to be introduced into the cells is provided to the cells, for example by means of topical administration, and a field is applied by electrodes 110 and 112, potentials on the electrodes being provided and controlled by a device 130, optionally under the control of a programmable unit 132. The electrodes are shown as being external electrodes distanced from the body, though any form or location of electrodes as disclosed herein may be used. The field may be re-applied at intervals.

In a further embodiment, the first composition 120 is administered as above, a chosen time interval t1 is allowed to elapse, and then a second composition 122 comprising a second particle type is administered. A second time interval t2 is then allowed to elapse, and the field is applied as described above.

In preferred embodiments the first particle type is adapted to enter cells (either all cells, or target cells selectively) as described above and the second particle type (where used) is adapted to bind selectively to the exterior of target cells but not to non-target cells. In an alternative embodiment the first and second composition both comprise the same particle type, adapted either to enter the target cell selectively or to bind selectively to the exterior of the target cell. In some embodiments the particles are adapted to remain in position within or associated with the target cells for a period of time within which multiple applications of the electric field may be made.

FIG. 11d shows a cross section through a body 102 during treatment of target cells in a region 100 of the body. Electrodes 110 and 112 in position A apply a field in direction E1. One or both electrodes may be in contact with the body as shown for electrode 110, or separated from it as shown for 112. Electrode 112 is shown also in an alternative position B that provides a field in a second direction E2. One or both electrodes may be made movable, for example by a clinician or automatically, moved by a motor means (not shown) under the control of the device 130 or programmable unit 132.

FIG. 11e shows a cross section through a body 102 during treatment of target cells in a region 100 of the body. Electrodes 110 and 112 apply a field. Electrode 112 is movable in x and y directions parallel to the skin surface, and may be for example a hand-held probe, and may be separated from the skin by an air gap or in contact with it, separated by an insulating layer 116.

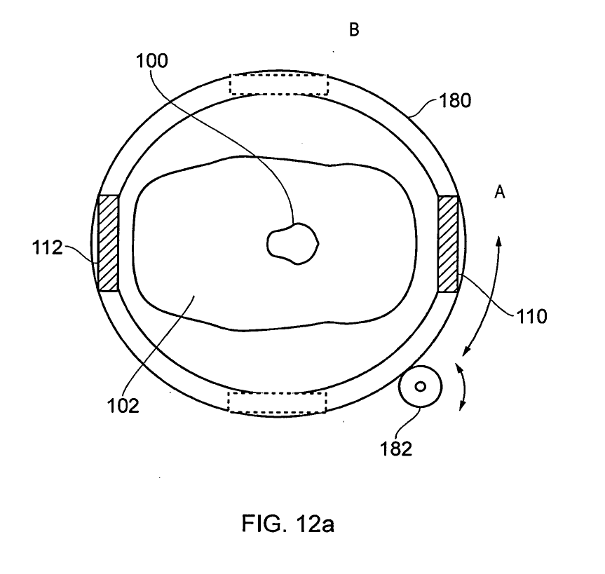

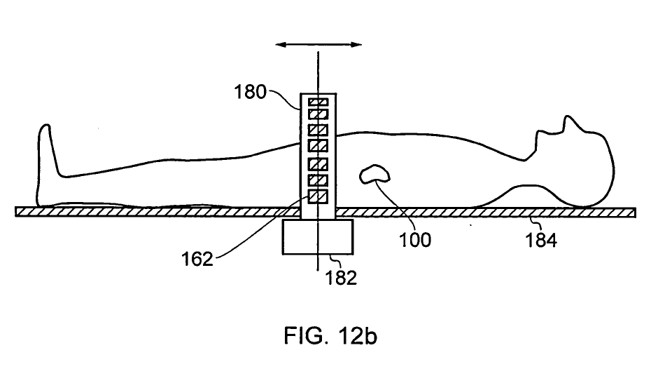

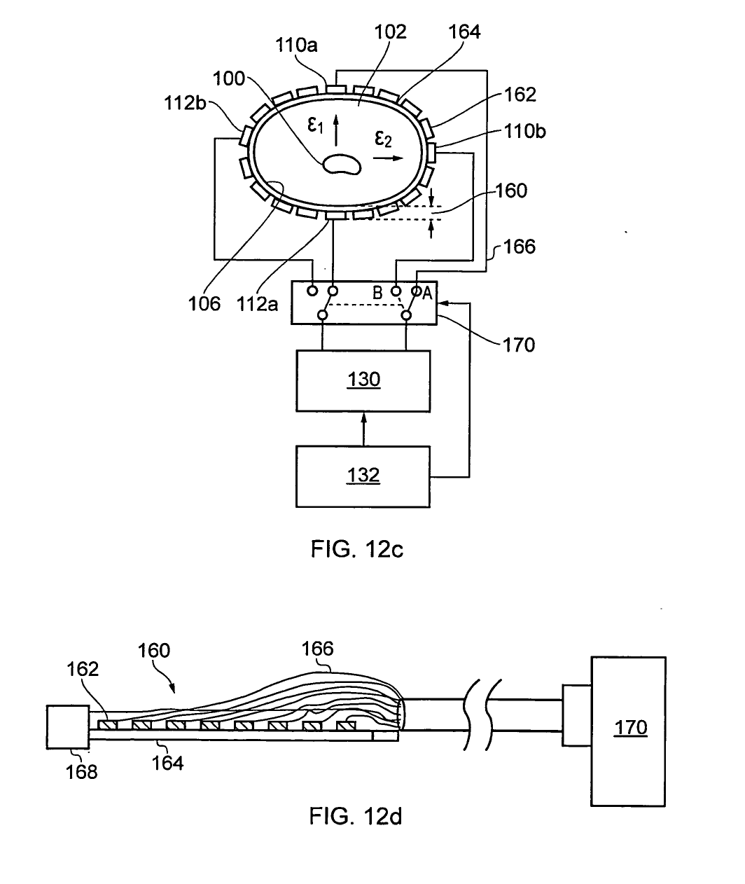

FIG. 12a shows an embodiment of an apparatus usable with the method of the invention and as part of the apparatus of the invention, for the treatment of disease in a subject, for example by electroporation of target cells within a region 100 of a body 102. Electrodes 110 and 112 are mounted in a structure 180 that supports them separated from and close to the body, and is adapted to cause them to rotate around the body as shown by the arrows, so moving them through a range of orientations with respect to the body. This allows the applied field between them to be moved through a range of orientations with respect to the region 100 and the target cells and particle arrangements, within it.

For example, the structure 180 might be moved by a motor means 182 coupled to it between a first position A and a second position B, so moving the direction of the applied field through an angle, say 90 degrees. The angle of rotation might be smaller or greater than 90 degrees, and may be chosen according to the location and nature of the region 100, for example it may be up to 180 degrees in one sense or both senses. More than one pair of electrodes may be provided within the structure 180, the pair that is providing the field being selected by a switch means, in order further to control the field direction at any time or point in the treatment process. The structure 180 might rotate about the body 102 in a horizontal plane, e.g. while the subject is standing, or in a vertical plane, e.g. when the subject is lying flat.

FIG. 12b shows a further embodiment of an apparatus usable with the method of the invention and as part of the apparatus of the invention, for the treatment of disease in a subject. Here two or more electrodes 162 (a plurality are shown) are provided within a structure 180 that is now adapted to move in a direction along the body of a subject, shown here as lying flat on a treatment surface or table 184, driven for example by motor means 182. At least one pair of electrodes within the structure 180 are have potentials applied to them so as to generate a field between them as described further for FIG. 12c. The structure 180 may then move along the body of the subject in order to subject a range of target cells within the body to the field. It will be apparent that the structure 180 may also rotate around the body as shown in FIG. 12a to produce a combined motion and a combined range of electric field directions. Motion in one or both dimensions may be controlled by a control means provided as part of the device of the invention or the programmable unit. In this way target cells may be exposed to a field in both of two dimensions. A further structure 180 (not shown) may be provided separated laterally from and parallel to the first in order to provide control a component of field orientated in the third dimension. Such an apparatus is applicable in cases where target cells are not all localised within a region 100, allowing treatment of large parts of the body without the use of large electrodes.

FIG. 12c shows a further embodiment of an apparatus usable with the method of the invention and as part of the apparatus of the invention, for the treatment of disease in a subject, for example by electroporation of target cells within a region 100 of a body 102. Here two or more electrodes are provided as part of an electrode structure 160, which is preferably flexible and may be shaped to, placed around or attached to the body or a body part, for example in the manner of a belt or armband, the electrodes themselves preferably being separated by a thin layer of insulator from the skin 106, but in some embodiments at least one electrode being in contact with it.

The electrodes are mounted on a flexible substrate 164 and are connected by leads 166 by means of switch unit 170 to the device 130 and programmable unit 132. The switch unit in use serves to connect pairs of electrode 110, 112 to the device so as to provide the field. As different pairs around the structure 160 are connected, so the field orientation is changed. The switch unit may be controlled by the device or the programmable unit to provide a desired pattern of field orientations during a treatment, shown as for example E1 when electrodes 110a and 112a are connected by the switch in position A, and E2 when electrodes 110b and 112b are connected by the switch in position B.

A preferred embodiment of the apparatus may be portable and worn as a belt and is usable with the method of the invention.

FIG. 12d shows the electrode structure 160 laying flat, optionally provided with a fastening mechanism 168 to fasten the structure to or around the body for example in a semi-rigid configuration or in the manner of a flexible belt. Electrode connections are shown loose for clarity; these are preferably in the form of a multiway cable.

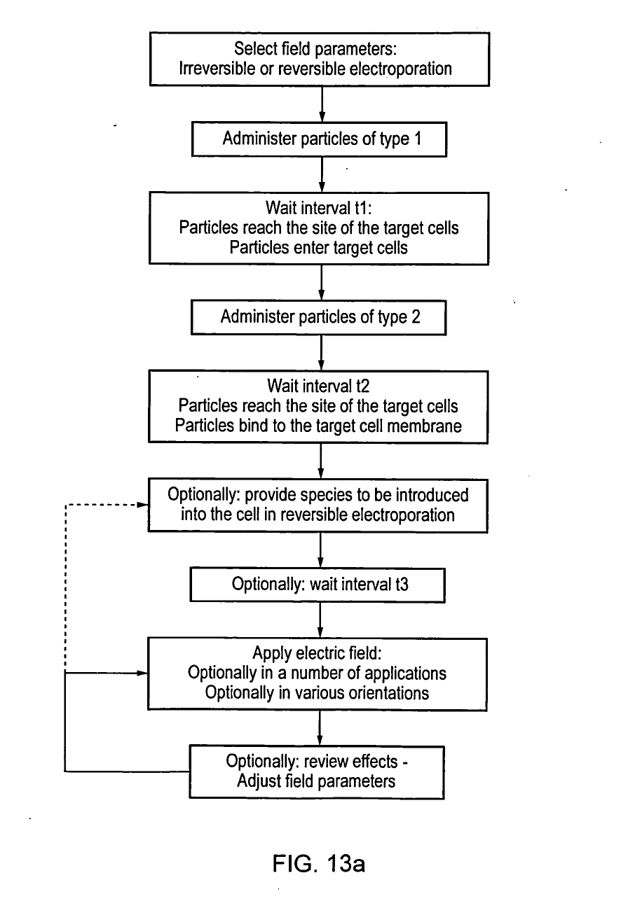

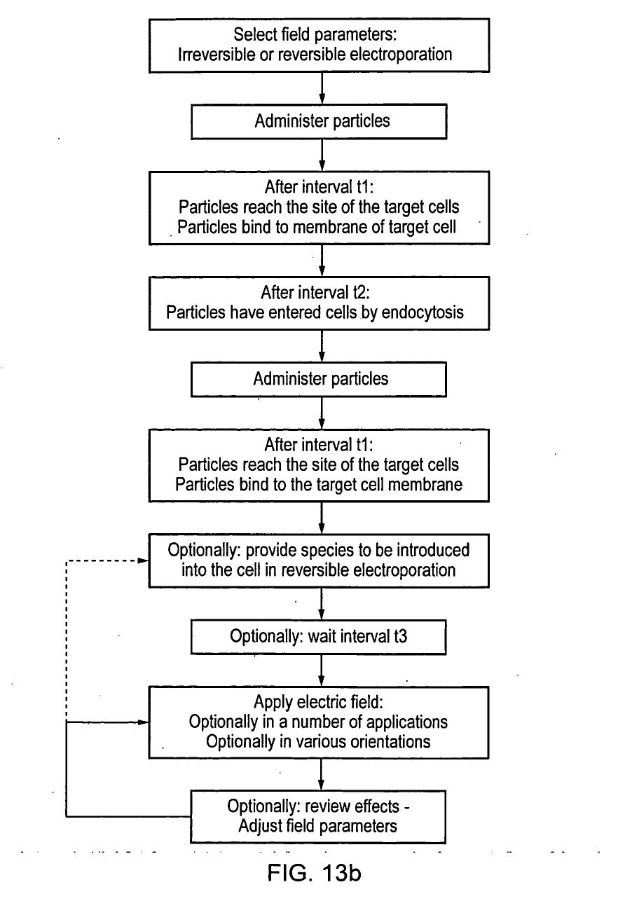

FIG. 13a shows a flow diagram for a method according to the invention, for the embodiment where a first particle type is adapted to enter the target cells and a second particle type is adapted to bind to the surface of the target cells. In embodiments directed to reversible electroporation, a species to be introduced into the target cells may be administered along with particles of type 2, and may be bound to the surface of the particles of type 2. If the species is administered separately from the particles a wait interval t3 is preferably allowed for the species to reach the vicinity of the target cells. It is envisaged that multiple applications of the electric field may be made following provision of the particles.

FIG. 13b shows a flow diagram for a method according to the invention for the case where only a single particle type is used, the particle being adapted to enter the target cell selectively, for example by targeted association with the surface of the target cell before endocytosis as described previously. In this embodiment the location and distribution of the particles is controlled by the timing of administration according to the method.

Particles from the first administration are allowed time interval t1 to bind the target cell surface and then be taken into the target cell. After interval t1 the second administration is then made and interval t2 allowed to elapse, to allow the second group of particles to bind to the exterior of the target cells, but t2 is not long enough to allow the particles substantially to be taken into the cells. In embodiments directed to reversible electroporation, the species to be introduced into the cells may be administered along with the second administration, or subsequently, with an optional wait interval t3. The field is then applied. In general in this method t2 is less than t1.

Optionally the methods as shown in FIGS. 13a and 13b may include steps of imaging the region 100 to determine the location and number of particles, and hence the readiness for application of the field. Imaging may also be used to determine the effectiveness of the treatment. Preferred embodiments of the method include the steps of monitoring the effectiveness of electroporation, or of the treatment resulting from electroporation, and adjusting the parameters of the applied field accordingly.

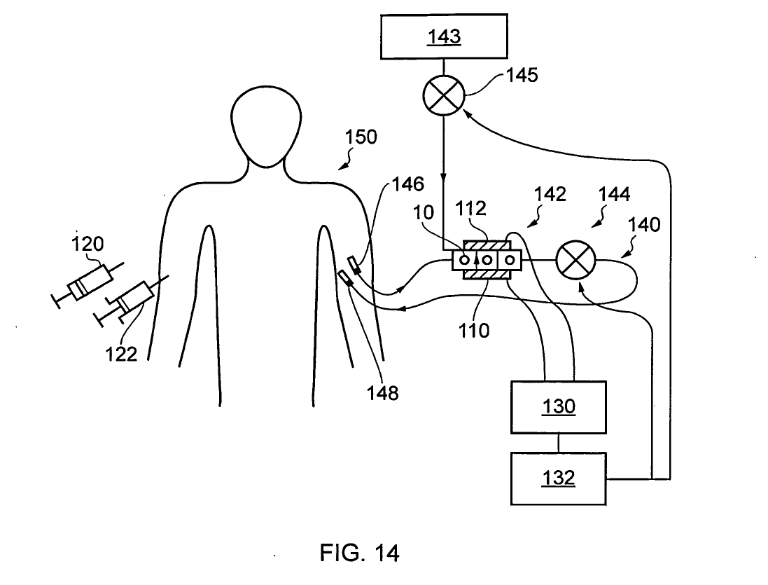

FIG. 14 shows a method and an apparatus for treatment of a disease within a subject by means of electroporation of target cells suspended in the subject's blood. A composition 120 comprising a first particle type is administered systemically to a subject 150 for example by means of injection or infusion into the blood stream, and particles then associate with target cells within the blood. After a chosen time interval t1, chosen to allow particles to reach the target cells and form desired particle arrangement(s) with respect to the target cells, either internal to the cell or bound to the exterior or both. Blood is drawn from the circulation by means of a first cannula 146 and flowed through an extracorporeal flow circuit 140 comprising a flow cell 142 and preferably controlled by a pump or flow control means 144.

In a preferred embodiment directed to reversible electroporation the species to be introduced into the target cells is added to the flow by means of a reservoir 143 and a flow control means such as a pump 145. A field is provided in the flow cell by electrodes 110 and 112 disposed on either side of the flow cell, such that target cells comprising particles are electroporated by the method of the invention on passing through the flow cell and the species introduced into them, non-target cells being substantially unaffected. Blood is then flowed back to the subject through a second cannula 148.

In a preferred embodiment a second composition 122 comprising a second particle type is administered after the interval t1, and a further interval t2 allowed to elapse; the blood is exposed to the field, the first and second particle types being adapted as described previously.

The method may be carried out by an apparatus comprising elements as shown in FIG. 14, namely standard cannulae and extra-corporeal blood flow components and pump or flow control means. Flow cells for conventional electroporation are known. The flow cell forming part of the invention may take any form adapted to allow a sufficient flow rate of blood while applying an electric field to the flow, and may be substantially planar or tubular, for example formed from concentric cylinders. A suitable flow cell comprises two parallel surfaces separated by a flow space, a substantially planar electrode mounted on or disposed above each surface. Electrodes are preferably insulated from the blood in the flow space. The dimensions of the flow cell, the flow rate through the cell, the field intensity profile are all chosen to give effective electroporation of the target cells while minimising damage to the target cells and minimising electroporation of non-target cells. Flow may be continuous or may be intermittent. Flow, addition of the species and the field pulse profile may be controlled by the device 130 or programmable unit 132.

In a further embodiment the method further comprises the step of fractioning the blood and applying the method of the invention to a blood fraction that contains the target cells. In this embodiment blood is withdrawn from the subject and processed by conventional apheresis techniques, the desired fraction being withdrawn from the apheresis process to be treated using the method and apparatus as described above.

In a further embodiment, electroporation of target cells in blood may be carried out in-situ within the body of the subject by providing particles as above and applying an electric field to a region of the circulatory system through which the target cells pass, carried in the blood. Flexible electrode apparatus similar to that shown in FIG. 12c may attached to the body and used to apply a field to for example a region of blood vessel or other perfused area, so as to effect gradual electroporation of target cells as they pass through the field.

The invention has applications in biological processes, for example in cell culture. By selectively electroporating target cells and leaving non-target cells unaffected, target cells within a mixed population can be identified and treated. By selectively destroying target cells and leaving non-target cells unharmed, a mixed population can be purified of the target cells, for example when these are a minority contamination, or if the non-target cells are a desired minority cell type, the population can be enriched in non-target cell type by selective destruction of the target cells. Such methods have application in for example production of a cell therapy product, in identifying desired cells from a mixed population, identifying contaminating cells, or carrying out genetic manipulation of a set of target cells in mixed population; removing target cells, for example removing residual undifferentiated target cells from a population of desired differentiated cells, for example by means of IRE or by means of introducing an apoptosis-inducing substance; or for selecting a non-target cell for further expansion by selectively identifying or removing the majority target cells from culture.

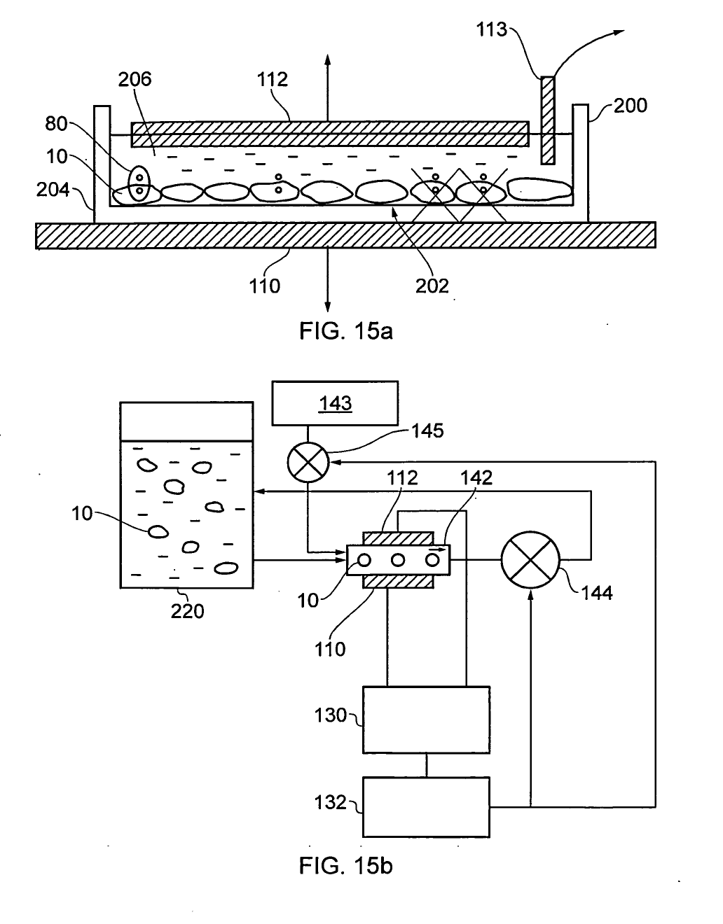

FIG. 15a shows an embodiment of the apparatus of the invention, adapted for use in a biological process, here the cell culture of attached cells in an apparatus 200. In FIG. 15a cells 10 are cultured on a surface 202 within a container 204, in contact with a medium 206. Particles are added to the medium and allowed to associate with the cells in a manner as described for previous embodiments. The species to be introduced into the target cells is added to the medium. A field is then applied across one or more cells, in preferred embodiments across the thickness of the cell layer as shown, either over the whole region of the cell layer, or over specific sub-regions.

The field is chosen so that target cells undergo electroporation or, according to the embodiment, non-thermal cell death resulting from the field exposure, for example by irreversible electroporation, mediated by the particles, while non-target cells are unaffected or affected to a lesser degree. The field may be applied in some embodiments by a first planar electrode 110 and second planar electrode 112. A first electrode might be mounted on or formed as part of the container 204. The second electrode is then preferably moveable or removable so as to access the interior of the container. The second electrode may be in contact with the medium, and may be insulated from it. Alternatively, the second electrode may be in electrical connection with the medium. The second electrode may alternatively be a probe-type electrode 113, which may be in electrical contact with the medium, the medium in some embodiments being conductive so as to provide a common potential over the upper surface of the cell layer.

Alternatively the probe electrode may be insulated from the medium, and may be positionable over a region of the culture or over a single cell to localise the effect of the field. Such a probe electrode may be formed for example from a wire, or a tube or glass pipette filled with conducting liquid, and in one embodiment may contain the species to be introduced into the cell. While a single surface and a single layer of cells is shown in FIG. 15a, the invention is applicable to other known forms of container with single or multiple culture surfaces.

FIG. 15b shows a further embodiment of an apparatus according to the invention, adapted to apply the invention to cell culture in suspension. Cells are cultured in suspension in a bioreactor 220. Particles are mixed with cells in medium, flowed through a flow cell 142 in which they are exposed to a field. Target cells are electroporated or destroyed selectively as described previously. The suspension comprising treated target cells may then be returned to the bioreactor, or alternatively to a second bioreactor (not shown).

Particles may be added to the medium in the bioreactor, or in the flow system outside the bioreactor. An incubation time may be provided to allow particles to associate with the target cells. In reversible electroporation, a species to be introduced into the target cells may be present in the bioreactor, or added to the flow path entering the flow cell. A suitable flow cell may be as described with respect to FIG. 14, for example a planar structure with electrodes on either side of a flow space, or a tubular structure with a concentric cylindrical electrode arrangement. More than one flow space may be provided in parallel, each with a pair of electrode surfaces one each side of the space, for example a stack of electrode pairs with flow spaces between them, with common inlet and outlet manifolds.

The electrode potentials are provided by a device 130 under control of a programmable unit 132. Flow may be continuous or intermittent, so providing a batch process. Flow may be provided by a pump or flow controller 144, preferably under control of a control means associated with the device 130 or (as shown) the programmable unit 132. Species to be introduced into the cells by means of reversible electroporation may be added from a reservoir 143, by means of flow control means 145.

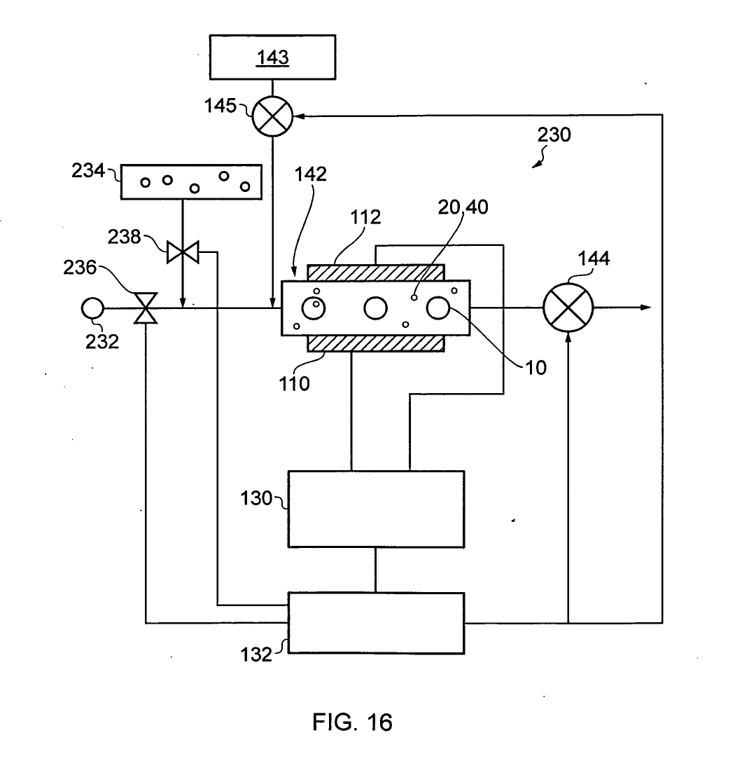

FIG. 16 shows an apparatus adapted to apply the method of the invention to sample processing, for example in diagnostics, the invention providing an improved means of cell labelling to allow identification of cell types or cell contents or components, for example biomarkers present within the cell whose identification relies on a marker substance or particle being introduced through the cell membrane.

A fluidic system 230 is provided comprising an inlet port 232, a flow cell 142 having means to apply a field to the contents of the flow cell, a supply of particles 234 connected to the flow system, and a flow means such as a pump 144. A sample containing cells is drawn through an inlet port 232, particles are mixed into a sample from a reservoir 234, allowed to associate with the cells, and then a field is applied in a flow cell 142. Alternatively, cells may be pre-incubated with the electroporation mediator particles. The field is provided by electrodes associated with the flow cell, controlled by the device 130 and the process may be controlled by a control means associated with the programmable unit 132, controlling the pump 144 and valves 236 and 238. A supply of the species to be introduced into the cell may be provided in a reservoir 143, connected to the flow system, and a flow means such as a pump 145. A sample is drawn through an inlet port 232, the marker particles are mixed into a sample from a reservoir 234, allowed to associate with the cells, and then a field is applied in a flow cell 142. The field is provided by electrodes associated with the flow cell, controlled by the device 130 and the process may be controlled by a control means associated with the programmable unit 132, controlling the pump 144 and valves 236 and 238. The apparatus and method may be applied to lysing of all cell types within the sample by using particles adapted to associate with a wide range of cell types, or to lyse target cells selectively be means of targeted particles as described above.

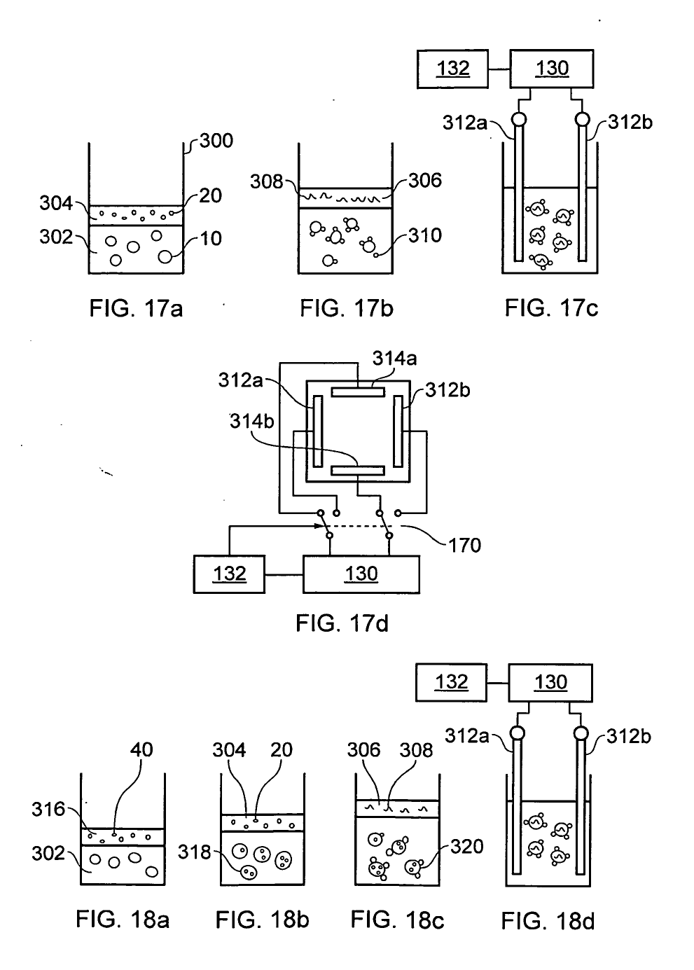

FIG. 17 shows a further embodiment in which the method of the invention is applied to a cell suspension in a vessel such as an electroporation cuvette. It is known in the art to use electroporation to introduce species into cells in suspension, for example to transfect DNA constructs into the cells. Often the species to be introduced is precious or available in small quantity, so it is advantageous to use a high concentration of cells in order to minimise the volume surrounding the cells into which the species is mixed. Problems arise from the non-uniform field conditions experienced by the cells, for example the screening of cells from the field by neighbouring cells, which means that high fields are needed to achieve an overall high electroporation efficiency, with the consequent problem of damage to the proportion of the cells exposed to a higher effective field. The method and apparatus of the invention provides an improved means to achieve electroporation of cells in a suspension using lower field strength and hence reducing the chance of such damage.

The steps in the method are shown in FIGS. 17a-17d and 18a-18d. In FIG. 17a a suspension 302 of target cells 10 is placed in a vessel 300 such as an electroporation cuvette. A suspension 304 of particles 20 adapted to associate with the exterior of the membrane of cells 10 is added to the vessel and mixed in, an interval t2 being allowed for the particles to associate with the cells. In FIG. 17b a suspension or solution 306 comprising species 308 to be introduced into the cell, shown here diagrammatically as a DNA construct as used in transfection, is added to the vessel and mixed in. As shown in FIG. 17c species 308 is then introduced by electroporation, by means of a field applied by electrodes 312a, 312b, powered by a device 130 adapted to provide a time varying electrical potential to the electrodes, preferably under the command of a control means 132 as described previously. The electrodes may be in contact with the liquid medium in the vessel, may be insulated from it, may be situated outside the vessel, or may form part of the walls of the vessel, the reduced electric field enabled by the method of the invention allowing a greater choice of configuration of the electrodes, and lower applied potentials, than in the prior art. As shown in FIG. 17d, more than one pair of electrodes may be provided in the vessel, which may be adapted to provide electric fields at a number of orientations, in order to overcome any sensitivity of the particle/cell arrangements to the field direction as described herein. Switch means 170 may be provided to select the electrode pairs in use, and so the field orientation, preferably under the control of the control means 132, allowing fields in a number of orientations to be used as part of a single electroporation procedure. In an alternative embodiment stirring means may be provided within the vessel 10 to change the orientation of the particle/cell arrangement between successive electroporation pulses applied by the device 130. The stirrer is preferably under the control of the control means 132.

In FIGS. 18a to 18d the method of the invention using a first particle type adapted to enter the cell and a second particle type adapted to associate with the exterior of the cell membrane is shown. In FIG. 18a a suspension 302 of target cells 10 is placed in a vessel 300 such as an electroporation cuvette and a suspension 316 of particles 40 adapted to enter the target cells 10 is added to the vessel and mixed in, an interval t1 being allowed for the particles to associate with and then enter the cells to produce a population of particle loaded cells 318. In FIG. 18b a suspension 304 of particles 20 adapted to associate with the exterior of the membrane of cells 10 is added to the vessel and mixed in, an interval t2 being allowed for the particles to associate with the cell membrane to produce a population of cells 320 having a set of arrangements of two or more particles associated with the cells, at least one particle being inside the cell and one associated with the exterior of the cell membrane. In FIG. 18c a suspension or solution 306 comprising species 308 to be introduced into the cell, shown again as a DNA construct, is added to the vessel and mixed in. As shown in FIG. 18d species 308 is then introduced into the cells by electroporation, by means of a field applied by electrodes 312a, 312b, powered by a device 130; according to the embodiment multiple electrodes and/or stirring may be arranged as described above.

Particles may be targeted to target cells by means of ligands adapted to bind to target molecules, for example cell receptors. Receptor-mediated endocytosis is known, in which particles bound by ligands to cell receptors are engulfed by the cell. Particles may be adapted by means of size, shape and ligand coating to be taken into cells by endocytosis or to be taken in to a lesser extent while remaining bound to the cell surface. For suitable particle sizes and morphology see for example Decuzzi and Ferraro US2010/029785; Zhang et al. Adv. Mater. 2009 vol. 21 p. 419-424; Muro et al. Molecular Therapy 2008 vol. 16 p. 1450-58.

In preferred embodiments of the invention, particles are used that provide suitable values of the following properties: rate of binding to a target cell membrane; affinity of binding; rate of endocytosis and rate of degradation of particles by lysosomes once inside the cell. These will depend in general on the size, shape and material of the particle and the nature and coverage of the ligands forming part of the coating.

Embodiments may comprise particles of types adapted to suit the target cell type, the target molecule, and the mode of administration and may comprise particles of dimensions chosen from a range. Particles below 0.1 um in diameter are taken up readily by endocytosis and may be taken up in large numbers by target cells. Some embodiments of the invention comprise particles that in use tend to provide up to around ten particles within a target cell; in other embodiments up to around several tens, or in further embodiments up to around several hundred or over 1000 particles within a target cell.

Larger particles up to around 1 um diameter may be taken up in smaller numbers, and some embodiments comprise particle types that in use tend to provide up to around 10 particles in a cell, in further embodiments 1 to 5 particles. Particles of greater than 1 um diameter may also be taken up in small numbers by a cell and may be used in some embodiments. Preferably particles are adapted to control the rate of endocytosis. For example, spherical particles undergo endocytosis more readily than larger aspect ratio, for example disc-shaped or elliptical particles, or rod-shaped particles.

In embodiments of the invention particles have a characteristic maximum dimension in the range 10 nm to 5 um. In a preferred embodiment at least one particle type has a maximum dimension of 0.1 um or less. In embodiments in which a single particle type is provided, adapted to associate with the exterior of the cell membrane, particles are selected having a characteristic dimension of from approximately 10 nm to 5 um. Preferably, a first particle type intended to be taken into the cell is of low aspect ratio, for example spherical, and small, for example 1 um or less in largest dimension, more preferably less than 500 nm and in some embodiments may be less than 100 nm in largest dimension, though larger and non-spherical particles are within the scope of the invention. In preferred embodiments where a second particle type is provided, intended to remain outside the cell membrane without promoting endocytosis, the second particle in some embodiments is large, for example greater than 50 nm in largest dimension, more preferably greater than 100 nm, and in some embodiments greater than 500 nm in largest dimension.

In a preferred embodiment one or both of a first and a second particle comprise a gold microsphere of order 1 um diameter. In a further preferred embodiment one or both of the first and second particle comprise a gold nanosphere 40 to 60 nm in diameter. In a preferred embodiment, at least a particle adapted to associate with the exterior of the cell membrane comprises a gold nanorod.

The first or the second particle types in some embodiments are non-spherical. For example, particles may be polyhedral, for example metal nanoprisms such as gold nanoprisms, elongated, for example elliptical, rod-like, tubular or may be formed from a cluster or agglomeration of smaller particles. A first particle adapted to enter the target cell and a second particle adapted to bind outside may be different sizes and different morphologies, preferably with the second particle having a larger maximum dimension and higher aspect ratio than the first. In a further preferred embodiment, at least a particle adapted to associate with the exterior of the cell membrane comprises a gold nanorod.

In preferred embodiments, the method of the invention comprises time interval(s) between the steps of the method, according to the nature of the particles in use, to allow sufficient time for the particles to reach their intended location and binding configuration but preferably not so long that internal particles suffer lysosomal degradation or particles intended to remain external undergo unwanted endocytosis.

Embodiments of the invention comprise particles formed from materials as described herein and materials similar to these as may be understood by the skilled person. Preferably particles comprise, or have a core comprising, a material that is a dielectric, ideally having a high relative permittivity, or that is conductive or semi conductive. Preferably particles comprise a material having a high permittivity or conductivity compared with the effective permittivity or conductivity of the environment or medium in which they are intended to be located in use (in this context, the terms medium and environment are used interchangeably herein). However, in embodiments, the invention is not limited to high permittivity or high conductivity particles, rather may derive its effect from the co-operative effects of lower permittivity or lower conductivity particles.

The material may have a permittivity selected from within a range according to the embodiment. A typical relative permittivity of a cell membrane is around 11.5 (see e.g. Raffa et al WO-A-2008/062378). Therefore in some embodiments a particle comprises a material having a relative permittivity of greater than or approximately equal to 11. In further embodiments a particle comprises a material having a relative permittivity of 20 or above, and in further embodiments greater than or approximately equal to that of blood or physiological saline (typical value 88). In further embodiments a particle may comprise a conductive material, for example having a semi conductive or conductive core, and in preferred embodiments the conductivity will be greater than or approximately equal to that of then environment in which it is intended to be located. Magnetic particles as used in magnetic separations comprise appropriate materials for use in embodiments of the invention, for example commercially-available magnetic separation particles, for example comprising a core of Fe3O4.

The invention ideally comprises particles, and apparatus and compositions containing particles, that may be selected by a skilled person based on the foregoing, together with choice of an appropriate field strength and waveform.

The term cell or target cell refers to a biological form of life comprising for example, a microorganism, a virus, or an eukaryote cell.

The eukaryote cell may e.g. be a plant cell, a plant spore, an animal cell such as mammal cell.

The mammal cell may e.g. be a human cell such as:

A keratinizing epithelial cell, such as a keratinocyte of epidermis, basal cell of epidermis (stem cell), keratinocyte of finger and toe nails, basal cell of nail bed (stem cell), hair shaft cell, hair root sheath cell, hair matrix cell (stem cell). Additionally a cell may be a wet stratified barrier epithelia such as, surface epithelial cell of stratified squamous epithelium of cornea, tongue, oral cavity, esophagus, anal canal, distal urethra, vagina or basal cell of these epithelia (stem cell), or cell of urinary epithelium (lining bladder and urinary ducts).

Other types of cell are an epithelial cell specialized for exocrine secretion such as cells of the salivary glands, cell of von Ebner's gland in tongue, cell of mammary gland, cell of lacrimal gland, cell of ceruminous gland of ear, cell of eccrine sweat gland, cell of apocrine sweat gland, cell of gland of Moll in eyelid, cell of sebaceous gland, cell of Bowman's gland in nose, cell of Brunner's gland in duodenum, cell of seminal vesicle, cell of prostate gland, cell of bulbourethral gland, cell of Bartholin's gland, cell of Littre's gland, cell of endometrium of uterus, isolated goblet cell of the respiratory and digestive tracts, mucous cell of the lining of the stomach, zymogenic cell of gastric gland, oxyntic cell of pancreas, Paneth cell of small intestine, type II pnemocyte of lung.

Other types of cell are cell specialized for secretion of hormones such as cells of anterior pituitary secreting growth hormone, follicle-stimulating hormone, luteinizing hormone, prolactin, adrenocorticotropic hormone, thyroid-stimulating hormone or cell of intermediate pituitary secreting melanocyte-stimulating hormone, or cell of posterior pituitary secreting oxytocin or vasopressin, or cell of gut and respiratory tract secreting serotonin, endorphin, somatostatin, gastrin, secretin, cholecystokinin, insulin, glucagon or bombesin.

Other types of cell are a cell of the thyroid gland secreting thyroid hormone or calcitonin, a cell of the parathyroid gland secreting parathyroid hormone or an oxyphil cell, a cell of adrenal gland secreting epinephrine, norepinephrine, steroid hormones such as mineralocorticoids or glucocorticoids, a cell of gonads secreting testosterone (Leydig cell of testis) estrogen (theca interna cell of ovarian follicle), progesterone (corpus luteum cell of ruptured ovarian follicle), a cell of juxtaglomerular apparatus of kidney secreting rennin, an epithelial absorptive cell in the gut, exocrine glands, and urogenital tract such as brush border cell of intestine (with microvilli), striated duct cell of exocrine glands, gall bladder epithelial cell, brush border cell of proximal tubule of kidney, distal tubule cell of kidney, nonciliated cell of ductulus efferens, epididymal principal cell or epididymal basal cell.

Other types of cell are a cell specialized for metabolism and storage such as hepatocyte, liver lipocyte or fat cell, an epithelial cell serving primarily a barrier function, lining the lung, gut, exocrine glands, and urogenital tract such as type I pneumocyte cell (lining air space of lung), pancreatic duct cell (centroacinar cell), nonstriated duct cell of sweat gland, salivary gland, mammary gland etc. (various), parietal cell of kidney glomerulus, podocyte of kidney glomerulus, cell of thin segment of loop of Henle (in kidney), collecting duct cell (in kidney), duct cell of seminal vesicle, prostate gland, etc. (various), an epithelial cell lining closed internal body cavities such as vascular endothelial cells of blood vessels and lymphatics (fenestrated, continuous, splenic), synovial cell (lining joint cavities, secreting largely hyaluronic acid), serosal cell (lining peritoneal, pleural, and pericardial cavities), squamous cell lining perilymphatic space of ear, cells lining endolymphatic space of ear, squamous cell columnar cells of endolymphatic sac (with microvilli or without microvilli), “dark” cell, vestibular membrane cell, stria vascularis basal cell, stria vascularis marginal cell, cell of Claudius, cell of Boettcher, choroid plexus cell (secreting cerebrospinal fluid).

Other types of cell are squamous cell of pia-arachnoid, cells of ciliary epithelium of eye (pigmented or nonpigmented), corneal “endothelial” cell, a ciliated cell with propulsive function of respiratory tract, of oviduct and of endometrium of uterus (in female), of rete testis and ductulus efferens (in male) or a cell of a central nervous system (ependymal cell lining brain cavities), a cell specialized for secretion of extracellular matrix such as epithelial ameloblast (secreting enamel of tooth), epithelial planum semilunatum cell of vestibular apparatus of ear (secreting proteoglycan), interdental cell of organ of Corti (secreting tectorial “membrane” covering hair cells of organ of Corti), nonepithelial (connective tissue) such as fibroblasts (various—of loose connective tissue, of cornea, of tendon, of reticular tissue of bone marrow, etc.), pericyte of blood capillary, nucleus pulposus cell of intervertebral disc, cementoblast/cementocyte (secreting bonelike cementum of root of tooth), odontoblast/odontocyte (secreting dentin of tooth), chondrocytes of hyaline cartilage of fibrocartilage of elastic cartilage, osteoblast/osteocyte, osteoprogenitor cell (stem cell of osteoblasts), hyalocyte of vitreous body of eye or stellate cell of perilymphatic space of ear.

Other types of cell are: a contractile cell such as skeletal muscle cells (red (slow), white (fast), inter-mediate, muscle spindle—nuclear bag, muscle spindle—nuclear chain, satellite cell (stem cell), or heart muscle cells (ordinary, nodal, Purkinje fiber), or smooth muscle cells (various), or myoepithelial cells of iris or of exocrine glands.

Other types of cell are a cell related to blood or the immune system such as red blood cell, megakaryocyte, macrophages and related cells (monocyte, connective-tissue macrophage (various), Langerhans cell (in epidermis), osteoclast (in bone), adendritic cell (in lymphoid tissues), microglial cell (in central nervous system)), neutrophil, eosinophil, basophil, mast cell, T lymphocyte (helper T cell, suppressor T cell, killer T cell), B lymphocyte (IgM, IgG, IgA, IgE), killer cell or stem cells and committed progenitors for the blood and immune system (various), a cell with sensory and transducing functions such as photoreceptors (rod, cones [blue sensitive, green sensitive, red sensitive], hearing (inner hair cell of organ of Corti, outer hair cell of organ of Corti), acceleration and gravity (type I hair cell of vestibular apparatus of ear, type II hair cell of vestibular apparatus of ear), taste (type II taste bud cell), smell (olfactory neuron, basal cell of olfactory epithelium (stem cell for olfactory neurons)), blood pH (carotid body cell [type I, type II]), touch (Merkel cell of epidermis, primary sensory neurons specialized for touch (various)), temperature (primary sensory neurons specialized for temperature [cold sensitive, heat sensitive]), pain (primary sensory neurons specialized for pain (various)), configurations and forces in musculoskeletal system (proprioceptive primary sensory neurons (various)).

Other types of cell are an autonomic neuronal cell (cholinergic (various), adrenergic (various), peptidergic (various)), a supporting cells of sense organs and of peripheral neurons such as supporting cells of organ of Corti (inner pillar cell, outer pillar cell, inner phalangeal cell outer phalangeal cell, border cell, Hensen cell), or a supporting cell of vestibular apparatus, or a supporting cell of taste bud (type I taste bud cell), or a supporting cell of olfactory epithelium, or a Schwann cell, or a satellite cell (encapsulating peripheral nerve cell bodies) or a enteric glial cell; a neuronal or glial cells of the central nervous system such as neurons (huge variety of types—still poorly classified), glial cells (astrocyte (various), oligodendrocyte).

It is appreciated that reference to cell includes the following cells: a lens cell such as anterior lens epithelial cell, lens fiber (crystallin-containing cell), or pigment cell such as melanocyte or retinal pigmented epithelial cell, a germ cell such as oogonium/oocyte, spermatocyte, or spermatogonium (stem cell for spermatocyte), a nurse cell such as ovarian follicle cell, sertoli cell (in testis), or thymus epithelial cell, Other types of cell are n interstitial cell such as interstitial cells of Cajal in the gastro-intestinal system, or interstitial cell of the kidney or other organs with pacemaker functions.

In embodiments the method of the invention is applied to electroporation of a cell membrane associated with a microorganism. The microorganism may e.g. be selected from the group consisting of an archeal microorganism, a eubacterial microorganism or a eukaryotic microorganism, the microorganism may be selected from the group consisting of a bacterium, a bacterial spore, a virus, a fungus, and a fungal spore.

In a preferred embodiment of the invention, the microorganism is hosted inside a mammalian cell which is serving as a reservoir for infection.

In a preferred embodiment of the invention, the microorganism is resistant to common chemotherapies such as anti-biotics such as but excluded too methicillin-resistant Staphylococcus aureus (MRSA), vancomycin-resistant Staphylococcus aureus (VRSA), penicillin resistant Streptococcus, anti-biotic resistant strains of Mycobacterium tuberculosis, penicillin resistant Enterococcus, multi-drug resistant Pseudomonas aeruginosa, clindamycin (or

[symbol]

What is claimed is:

1. A method of causing electroporation of a targeted cell comprising the steps of: causing at least one particle to become associated with the exterior of a target cell and exposing the target cell to an electric field for a sufficient duration in order to cause electroporation of the cell membrane, whereby in use, the particle has a higher permittivity than its surrounding environment.

2. A method according to claim 1 wherein the particle has a low aspect ratio, when compared with the target cell.

3. A method according to claim 1 wherein the particle is of substantially uniform dimensions.

4. A method according to claim 1 wherein the particle is substantially spherical.

5. A method according to claim 1 wherein characteristics of the electric field are selected whereby the magnitude of the electric field is in excess of 300V/cm such that cell experiences irreversible electroporation.

6. A method according to claim 1 wherein characteristics of the electric field are selected whereby the magnitude of the electric field is in the range 1V/cm to 500V/cm such that cell experiences reversible electroporation.

7. A method according to claim 1 wherein the particle comprises a material having a relative permittivity greater than approximately 11; preferably having a relative permittivity greater than approximately 88.

8. A method according to claim 7 wherein the particle comprises a material selected from the group including: a metal, a metal oxide, iron, an oxide of iron, silver, gold and platinum.

9. A method according to claim 1 further comprising the step of: causing at least one first particle to enter the cytoplasm of the target cell and at least one second particle to associate with the exterior of the cell membrane.

10. A method according to claim 1 wherein at least one particle comprises a coating selective for a target molecule on or within the target cell.

11. A method according to claim 1 wherein particles having a high permittivity or conductivity are administered to the body of a subject, the particles being adapted to associate with a target molecule on the target cell membrane or to be taken up within the target cell; allowing a chosen time interval to elapse so that at least one particle associates with or enters at least one target cell; and applying an electric field to a body region of the subject within which one or more target cells are located, in order to cause electroporation of the targeted cells.

12. A method according to claim 11 further comprising the steps of: administering to the subject, following said time interval, a second particle type so that at least one second particle is taken up within the cell; and allowing a second chosen time interval to elapse before applying the electric field.

13. A method according to claim 11 further comprising the steps of: administering to the subject, following said time interval, a second particle type so that at least one second particle is bound to its surface; and allowing a second chosen time interval to elapse before applying the electric field.

14. A method according to claim 13 wherein a target cell is in a liquid medium, such as a body fluid, and the electric field is provided in a region of the body fluid or through which the body fluid flows.

15. An apparatus for electroporation of target cells using particles, or nanoparticles, and a time-varying electric field, characterised in that a particle delivery means supplies particles to the target cells, the particles having a high permittivity or being conductive and being adapted to associate with a target molecule on or within the target cell, and, in use, a means exposes the target cells to an electric field sufficient to cause electroporation of the cell.

16. An apparatus according to claim 15, further comprising a first and a second electrode, at least one electrode being located external to the body of the subject, and a controller adapted to apply a variable potential to the first and second electrodes.

17. An apparatus according to claim 15 comprising means to associate particles with a target cell in a liquid medium, for example a body fluid, and means to expose particles in a liquid medium to a field so as to cause electroporation of the cells.

18. An apparatus to expose target cells to a variable electric or electromagnetic field comprising: particles adapted to enter a target cell, at least a first and a second electrode and a device comprising a controller, operating under control of instructions in the form of software and using data derived from a look-up table, wherein the device applies a variable potential to the first and second electrodes, in accordance with the data and under instruction from the software, whereby in use, target cells within the electric field are killed and non-target cells remain substantially unharmed.

19. A method of establishing an electric field, proximal to a cell wall of a cell within a host comprising the steps of: providing a particle within the interior, or attached to the exterior of a cell, providing a second particle within the interior, or attached to the exterior of a cell, establishing an applied electric field within the cell host by means of a first and a second electrode external to the host, thereby generating an electric field capable of effecting cell death by non-thermal means, for example irreversible electroporation.

20. A composition comprising: a plurality of particles adapted for use in a method and system for causing electroporation of a target cell, the particles being adapted to associate with target molecules on or within the target cells and adapted to cause an enhancement of an applied electric or electromagnetic field in their vicinity.

21. A composition according to claim 20 comprising particles adapted to associate selectively with a target molecule on the target cell membrane.

22. A method for a microbiological process, the method comprising the steps of: providing a number of at least a first particle type to cells in culture, the cells comprising target cells, the particles adapted to associate selectively with target cells; allowing the particles either to bind to target molecules on the surface of the target cells or to be taken up inside the target cells; and applying an electric field to the cells in culture, so causing electroporation of target cells.

23. An analytical process comprising the steps of: providing a plurality of a first particle type to cells in a liquid sample; allowing the particles either to bind to target molecules on the surface of the cells or to be taken up inside the cells; and applying an electric field to the liquid sample, so causing electroporation of the cells.
